(19) United States
(12) Patent Application Publication
(10) Pub. No.: US 2022/0211663 A1
(43) Pub. Date: Jul. 7, 2022

(54) NANO CO-DELIVERY OF QUERCETIN AND ALANTOLACTONE PROMOTES ANTI-TUMOR RESPONSE THROUGH SYNERGISTIC IMMUNOGENIC CELL DEATH FOR MICROSATELLITE-STABLE COLORECTAL CANCER

(71) Applicant: The University of North Carolina at Chapel Hill, Chapel Hill, NC (US)

(72) Inventors: Leaf Huang, Chapel Hill, NC (US); Jing Zhang, Chapel Hill, NC (US); Limei Shen, Chapel Hill, NC (US)

(21) Appl. No.: 17/605,371

(22) PCT Filed: Apr. 23, 2020
(86) PCT No.: PCT/US2020/029448

§ 371 Date: Oct. 21, 2021

Related U.S. Application Data
(60) Provisional application No. 62/837,536, filed on Apr. 23, 2019.

(51) Int. Cl. A61K 31/365; A61K 31/352; A61K 9/107; A61K 47/22; A61K 47/10; A61K 47/24; A61P 35/00

(52) U.S. Cl. CPC A61K 31/365; A61K 31/352; A61K 9/1075; A61P 35/00; A61K 47/10; A61K 47/24; A61K 47/22

(57) ABSTRACT

Disclosed are micellar formulations comprising a synergistic combination of quercetin and alantolactone and their use for treating a cancer, including microsatellite-stable colorectal cancer (CRC), which otherwise is resistant to immunotherapy. The combination of quercetin and alantolactone was found to induce synergistic immunogenic cell death (ICD) at synergistic ratiometric micellar loadings.

### FEDERALLY SPONSORED RESEARCH OR DEVELOPMENT

This invention was made with government support under Grant Number CA198999 awarded by the National Institutes of Health. The government has certain rights in the invention.

### BACKGROUND

Colorectal cancer (CRC) is a one of the leading causes of cancer death throughout the world, affecting both women and men. It is the third major cause of death in U.S. Surgical resection, chemotherapy (e.g., CapeOX, FOLFOX, or FOLFIRI), and radiotherapy are standard clinical treatments. These regimens, however, are not effective for the advanced stage of the disease as a high recurrence rate after surgery is still troublesome. In 2017, more than 95,520 new cases of colon cancer and about 40,000 new cases of rectal cancer were reported in the U.S. alone. Although colonoscopy and other screening and preventative measures improve the survival rates, less than 40% of CRC can be diagnosed at a localized stage. The five-year survival rate dramatically falls from 90% at the local stage to only 14% when the cancer metastasis occurs, for example, in the liver.

In recent years, treatment approaches based on modulating the immune system have been successful in treating a variety of cancers. These approaches include immune blockade inhibitors that interfere with the programmed death-1/programmed death-ligand 1 (PD-1/PD-L1) to overcome immune suppression or chimeric antigen receptor T-cell therapy by engineering patient T-cells to recognize and attack cancer cells. In CRC, patients with defective DNA mismatch repair system (MMR) or microsatellite instability (MSI-H) are more responsive to immunotherapy. Only between 5%-15% of patients display MMR-deficient/MSI-H. Checkpoint blockade immunotherapies could be very effective for patients whose tumors are pre-infiltrated by T cells. Unfortunately for colorectal cancer patients, about 95% of the patient population does not respond to the PD-1/PD-L1 blockade treatment.

### SUMMARY

In some aspects, the presently disclosed subject matter provides a micellar formulation comprising a synergistically effective amount of quercetin and alantolactone, or derivatives thereof, for treating a cancer. In particular aspects, the quercetin and alantolactone are present in the micellar formulation in a molar ratio selected from the group consisting of about 1:13 quercetin:alantolactone (mol/mol), about 1:7 quercetin:alantolactone (mol/mol), and about 1:4 quercetin:alantolactone (mol/mol). In yet more particular aspects, the quercetin and alantolactone are present in the micellar formulation in a molar ratio of about 1:4 quercetin:alantolactone (mol/mol). In certain aspects, the micellar formulation comprises a combination of 1,2-distearoyl-sn-glycero-3-phosphoethanolamine-N-methoxy-poly(ethylene glycol 2000) (DSPE-PEG2000) and D-α-Tocopherol polyethylene glycol succinate (TPGS).

In other aspects, the presently disclosed subject matter provides a method for treating a cancer in a subject in need of treatment thereof, the method comprising administering to the subject a therapeutically effective amount of a micellar formulation comprising a synergistically effective amount of quercetin and alantolactone to treat the cancer. In particular aspects, the cancer is selected from the group consisting of colorectal cancer, breast cancer, pancreatic cancer, cervical cancer, prostate cancer, and lymphoma. In yet more particular aspects, the colorectal cancer is microsatellite-stable colorectal cancer.

In certain aspects, administration of a synergistically effective amount of quercetin and alantolactone induces immunogenic cell death (ICD) and/or induces cancer cell apoptosis. In more certain aspects, administration of a synergistically effective amount of quercetin and alantolactone inhibits tumor growth and/or progression. In yet more certain aspects, the administration of a synergistically effective amount of quercetin and alantolactone reduces a percentage of immune cells in a tumor microenvironment of the cancer. In particular aspects, the immune cells in the tumor microenvironment of the cancer are selected from the group consisting of myeloid-derived suppressor cells (MDSCs) and T regulatory cells (Tregs).

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, and FIG. 1C show (FIG. 1A) Chemical structure of quercetin (Q) and alantolactone (A); (FIG. 1B) Immunogenic cell death (ICD) induced by Q or A alone and the combination of Q and A. High-mobility group box 1 (HMGB1)% positive cells as indicated by arrows were counted as positive green fluorescence overlapping with the red fluorescence; and (FIG. 1C) Combination index (CI) and IC₅₀ of Q and A on CT26-FL3 cells. ** p<0.005, * p<0.05, ns: not significant;

FIG. 2A and FIG. 2B show (FIG. 2A) ICD induced by Q or A alone and the combination of Q and A. HMGB1% positive cells as indicated by arrows were counted as positive green fluorescence overlapping with the red fluorescence; and (FIG. 2B) Morphology of QA-M;

FIG. 3A, FIG. 3B, FIG. 3C, FIG. 3D, FIG. 3E, FIG. 3F, and FIG. 3G show (FIG. 3A) Morphology of QA-M and its size and zeta potential. Bar indicates 50 nm; (FIG. 3B) Critical micellar concentration measurement of QA-M; (FIG. 3C) Particle size and entrapment efficiency (EE %) of QA-M micelles diluted from 12- to 60-fold with PBS buffer after 24 h incubation (pH 7.4) (n=3); (FIG. 3D) Cumulative release of Q from QA-M within 72 h at 37° C. in 100 mg/mL egg yolk lecithin suspension (n=3); (FIG. 3E) Pharmacokinetic curves of QA-F and QA-M at different time points after i.v. injection (n=6); (FIG. 3F) Micelle distribution in CT26-FL3 tumor-bearing mice at 24 h after injection with DiD-loaded micelles (150 μg/kg), and observed by IVIS imaging Region-of-interest (ROI) fluorescence intensities of tumors and major organs (n=3); and (FIG. 3G) Biodistribution of Q and A from QA-F and QA-M in tumors detected by UHPLC/MS at different time points after intravenous injection (n=3); ** p<0.005, * p<0.05;

FIG. 4A, FIG. 4B, FIG. 4C, FIG. 4D, and FIG. 4E show (FIG. 4A) Inhibition of tumor growth in different groups with Q at 3 mg/kg and A at 9 mg/kg (n=4). Tumors were collected at the end of experiment and weighted. Arrows indicate the days of injection; (FIG. 4B) Survival among different treatments, n=5; (FIG. 4C) ALT, AST, BUN and CREAT levels in PBS, Q-M, A-M and QA-M groups, n=3; (FIG. 4D) H&E staining of major organs and tumors in each group; (FIG. 4E) TUNEL positive cells (%) in tumors of each groups (n=4). **** p<0.0001, *** p<0.0005, ** p<0.005, * p<0.05;

FIG. 5A and FIG. 5B are (FIG. 5A) CT26-FL3 tumors were collected and imaged at the end of experiment with Q at 3 mg/kg and A at 9 mg/kg (n=4); and (FIG. 5B) Body weight changes of mice in CT26-FL3 tumor inhibition study (n=4);

FIG. 6. TUNEL positive cells (%) in tumors of each group (n=4). **** p<0.0001, * p<0.05;

FIG. 7A, FIG. 7B, FIG. 7C, and FIG. 7D show (FIG. 7A) Immunosuppressive cell population and relative mRNA expressions of cytokines in tumors of each group by flow cytometry and RT-PCR (n=4); (FIG. 7B) Cytotoxic T-lymphocytes and relative mRNA expressions of cytokines in tumors of each group by flow cytometry and RT-PCR, respectively (n=4); (FIG. 7C) Immunofluorescence staining and quantification of CD3⁺ cells in tumors of each group (n=4). Bar equals 100 μm; and (FIG. 7D) Western blot analysis and quantification of biomarkers in tumors of each group (n=3). **** p<0.0001, *** p<0.0005, ** p<0.005, * p<0.05, ns: not significant; ## vs QA-M, p<0.005, # vs QA-M p<0.05; ΦΦΦΦ vs PBS p<0.0001, ΦΦ vs PBS, p<0.005, Φ vs PBS, p<0.05;

FIG. 8 shows Treg cells and MDSCs in tumors of each group measured by flow cytometry (n=4). **** p<0.0001, ** p<0.005, * p<0.05, ns: not significant;

FIG. 9 shows TLR4⁺ and PD-L1⁺CD11c⁺ cells population in tumors of each group measured by flow cytometry (n=4). *** p<0.0005, ** p<0.005, * p<0.05, ns: not significant;

FIG. 10 shows cytotoxic T-lymphocytes in tumors of each group by flow cytometry (n=4). **** p<0.0001, *** p<0.0005, ** p<0.005, * p<0.05, ns: not significant;

FIG. 11 shows immunofluorescence staining and quantification of CD3⁺ cells in tumors of each group (n=4). Bar indicates 100 μm. **** p<0.0001, ** p<0.005, ns: not significant;

FIG. 12 shows western blot analysis and quantification of biomarkers in tumors of each group, n=3. ## vs QA-M, p<0.005, # vs QA-M p<0.05; ΦΦΦΦ vs PBS p<0.0001, ΦΦ vs PBS, p<0.005, Φ vs PBS, p<0.05;

FIG. 13A, FIG. 13B, and FIG. 13C show (FIG. 13A) Treatment scheme and tumor growth curves of CT26-FL3 tumors after the depletion of CD4⁺ and CD8⁺ cells. ** vs PBS group, p<0.005 (n=5); (FIG. 13B) Memory immune T cells in lymph nodes (LNs) analyzed by flow cytometry at the end of tumor-inhibition experiment. **** p<0.0001, ** p<0.005, * p<0.05, ns: not significant, (n=4); and (FIG. 13C) Tumor-bearing mice were subcutaneously inoculated with 4T1 and CT26-FL3 cells at each side of the body after total four injection of QA-M. Subcutaneous tumors were measured at list day after inoculation (n=5); ** p<0.005, ns: not significant;

FIG. 14 shows memory immune T cells in LNs analyzed by flow cytometry at the end of tumor-inhibition experiment (n=3). **** p<0.0001, *** p<0.0005, ** p<0.005, * p<0.05, ns: not significant; and

FIG. 15A, FIG. 15B, and FIG. 15C show (FIG. 15A) Tumor growth curve, (FIG. 15B) tumor images and (FIG. 15C) weight of 4T1 breast tumor treated by PBS, QA-F and QA-M every other day for total four injections (n=4). **** p<0.0001, *** p<0.0005, ** p<0.005, * p<0.05.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Nano Co-Delivery of Quercetin and Alantolactone Promotes Anti-Tumor Response Through Synergistic Immunogenic Cell Death for Microsatellite-Stable Colorectal Cancer

### A. Micellar Formulations Comprising a Synergistically Effective Amount of Quercetin and Alantolactone

In some embodiments, the presently disclosed subject matter provides a micellar formulation comprising a synergistically effective amount of quercetin and alantolactone, or derivatives thereof, for treating a cancer. Quercetin is a plant flavonol from the flavonoid group of polyphenols. Quercetin has the following chemical structure:

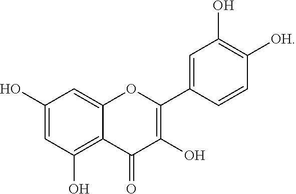 [SMILES: O=c1c(O)c(-c2ccc(O)c(O)c2)oc2cc(O)cc(O)c12]

Alantolactone is a sesquiterpene lactone that is found in many plant species and which has the following chemical structure:

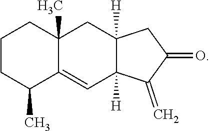 [SMILES: C=C1C(=O)C[C@@H]2C[C@@]3(C)CCC[C@H](C)C3=C[C@H]12]

As used herein, the terms “synergy,” “synergistic,” “synergistically” and derivations thereof, such as in a “synergistic effect” or a “synergistic combination” or a “synergistic composition” refer to circumstances under which the biological activity of a combination of quercetin (Q) and alantolactone (A) is greater than the sum of the biological activities of the respective agents when administered individually.

Synergy can be expressed in terms of a combination index (CI, which can be determined, for example, by using the Chou and Talalay method. Zhang et al., 2014; Chou et al., 1984. CI can be calculated by using the following equation (1):

CI=(D)₁/(Dₓ)₁+(D)₂/(Dₓ)₂ (1)

where (D)₁ and (D)₂ are the concentrations for a single drug after combination that inhibits x % of cell growth, and (Dₓ)₁ and (Dₓ)₂ are the concentrations for a single drug alone that inhibits x % of cell growth. CI values more than one demonstrate antagonism and CI values less than one demonstrate synergism of drug combinations.

In general, the lower the CI, the greater the synergy shown by that particular combination. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

In certain embodiments, the quercetin and alantolactone are present in the micellar formulation in a molar ratio selected from the group consisting of about 1:13 quercetin:alantolactone (mol/mol), about 1:7 quercetin:alantolactone (mol/mol), and about 1:4 quercetin:alantolactone (mol/mol). In particular embodiments, the quercetin and alantolactone are present in the micellar formulation in a molar ratio of about 1:4 quercetin:alantolactone (mol/mol).

In certain embodiments, the micellar formulation comprises a combination of 1,2-distearoyl-sn-glycero-3-phosphoethanolamine-N-methoxy-poly(ethylene glycol 2000) (DSPE-PEG2000) and D-α-Tocopherol polyethylene glycol succinate (TPGS). In certain embodiments, the micellar formulation comprises spherical particles. The spherical particle can have a diameter of less than about 150 nm, including but not limited to about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, and 150 nm. In particular embodiments, the particle has a diameter of about 15 to about 25 nm. In yet more particular embodiments, the particle has a diameter of about 20 nm.

In certain embodiments, the micellar formulation has a zeta potential of between about −1 to about −0.1 mV. In particular embodiments, the micellar formulation has a zeta potential of about −0.3±0.1 mV.

In certain embodiments, the micellar formulation has an encapsulation efficiency between about 80% to about 95%, including 80%, 85%, 90%, and 95%, for each of quercetin and alantolactone. In certain embodiments, the micellar formulation has an encapsulation efficiency of greater than about 90% for quercetin and alantolactone.

In certain embodiments, the micellar formulation has a critical micelle concentration (CMC) of about 0.003 mg/mL.

B. Methods for Treating a Cancer with Micellar Formulations Comprising a Synergistically Effective Amount of Quercetin and Alantolactone

In other embodiments, the presently disclosed subject matter provides a method for treating a cancer in a subject in need of treatment thereof, the method comprising administering to the subject a therapeutically effective amount of a micellar formulation comprising a synergistically effective amount of quercetin and alantolactone to treat the cancer. As used herein, the term “cancer” refers to or describe the physiological condition in mammals that is typically characterized by unregulated cell growth. As used herein, “cancer cells” or “tumor cells” refer to the cells that are characterized by bye this unregulated cell growth.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the term “inhibit,” and grammatical derivations thereof, refers to the ability of a presently disclosed compound, e.g., a presently disclosed compound of formula (I), to block, partially block, interfere, decrease, or reduce the growth of bacteria or a bacterial infection. Thus, one of ordinary skill in the art would appreciate that the term “inhibit” encompasses a complete and/or partial decrease in the growth of bacteria or a bacterial infection, e.g., a decrease by at least 10%, in some embodiments, a decrease by at least 20%, 30%, 50%, 75%, 95%, 98%, and up to and including 100%.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In particular embodiments, the cancer is selected from the group consisting of colorectal cancer, breast cancer, pancreatic cancer, cervical cancer, prostate cancer, and lymphoma. In yet more particular embodiments, the colorectal cancer is microsatellite-stable colorectal cancer. One of ordinary skill in the art would appreciate that other cancers could be treated by the presently disclosed methods, including, but not limited to, all forms of carcinomas, melanomas, sarcomas, lymphomas and leukemias, including without limitation, bladder carcinoma, brain mors, breast cancer, cervical cancer, colorectal cancer, esophageal cancer, endometrial cancer, hepatocellular carcinoma, laryngeal cancer, lung cancer, osteosarcoma, ovarian cancer, pancreatic cancer, prostate cancer, renal carcinoma and thyroid cancer. In some embodiments, the cancer to be treated is a metastatic cancer. In particular, the cancer may be resistant to known therapies.

In certain embodiments, administration of a synergistically effective amount of quercetin and alantolactone induces immunogenic cell death (ICD) and/or induces cancer cell apoptosis. In other embodiments, administration of a synergistically effective amount of quercetin and alantolactone inhibits tumor growth and/or progression. In yet other embodiments, administration of a synergistically effective amount of quercetin and alantolactone reduces a percentage of immune cells in a tumor microenvironment of the cancer. In certain embodiments, the immune cells in the tumor microenvironment of the cancer are selected from the group consisting of myeloid-derived suppressor cells (MDSCs) and T regulatory cells (Tregs).

In certain embodiments, administration of a synergistically effective amount of quercetin and alantolactone inhibits tumor-promoting inflammation in one or more cells. In other embodiments, administration of a synergistically effective amount of quercetin and alantolactone reduces Toll-like receptor 4 positive (TLR4⁺) expression in one or more cancer cells. In yet other embodiments, administration of a synergistically effective amount of quercetin and alantolactone reduces PD-L1 expression on one or more cancer cells.

In certain embodiments, administration of a synergistically effective amount of quercetin and alantolactone reduces secretion of immune-suppressive cytokines in one or more cancer cells. In particular embodiments, the immune-suppressive cytokines are selected from the group consisting of IL-10, TGF-β, IL-1β, and CCL2.

In certain embodiments, administration of a synergistically effective amount of quercetin and alantolactone activates one or more tumor-infiltrating immune cells in a cancer tumor. In particular embodiments, the one or more tumor-infiltrating immune cells comprises one or more CRT⁺ cells. In yet more particular embodiments, the one or more CRT⁺ cells are selected from the group consisting of a CD3⁺ T cell, a CD8⁺ T cell, and a CD4⁺ T cell.

In certain embodiments, administration of a synergistically effective amount of quercetin and alantolactone increases expression of a level of costimulatory signal (MHC class II and CD86) on one or more dendritic cells. In other embodiments, administration of a synergistically effective amount of quercetin and alantolactone increases a presence of natural killer (NK) cells. In yet other embodiments, administration of a synergistically effective amount of quercetin and alantolactone increases IFN-γ production from CD4⁺ and CD8⁺ T cells in a tumor comprising the cancer.

In certain embodiments, administration of a synergistically effective amount of quercetin and alantolactone activates T cells. In other embodiments, administration of a synergistically effective amount of quercetin and alantolactone induces higher levels of IL-12 and IFN-γ in a tumor comprising the cancer. In yet other embodiments, administration of a synergistically effective amount of quercetin and alantolactone increases the expression of CXCL9 in one or more cancer cells.

In certain embodiments, administration of a synergistically effective amount of quercetin and alantolactone increases the secretion of tumor necrosis factor alpha (TFN-α) in one or more cancer cells. In other embodiments, administration of a synergistically effective amount of quercetin and alantolactone down-regulates suppressive immune cells and cytokines. In yet other embodiments, administration of a synergistically effective amount of quercetin and alantolactone up-regulates immuno-active cells and cytokines.

In certain embodiments, administration of a synergistically effective amount of quercetin and alantolactone increases the expression of phosphor-AMP-activated protein kinase α (p-AMPKα) protein in one or more cancer cells. In other embodiments, administration of a synergistically effective amount of quercetin and alantolactone decreases the expression of mammalian target of rapamycin (mTOR) and phospho-mTOR (p-mTOR) in one or more cancer cells. In yet other embodiments, administration of a synergistically effective amount of quercetin and alantolactone inhibits Bcl-2 to induce cell apoptosis, thereby promoting autophagy. In even yet other embodiments, administration of a synergistically effective amount of quercetin and alantolactone produces p-AMPK and suppresses mTOR and p-mTOR, thereby promoting autophagy.

In certain embodiments, administration of a synergistically effective amount of quercetin and alantolactone activating innate immune response in tumors, thereby inducing the activation of an adaptive immune response and inhibiting tumor growth. In other embodiments, administration of a synergistically effective amount of quercetin and alantolactone recruiting tumor-specific memory T cells. In particular embodiments, the memory T cells include CD8⁺ and CD4⁺.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions. Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Nano Co-Delivery of Quercetin and Alantolactone Promotes Anti-Tumor Response Through Synergistic Immunogenic Cell Death for Microsatellite-Stable Colorectal Cancer

### 1.1 Overview

Microsatellite-stable colorectal cancer (CRC) is known to be resistant to immunotherapy. The combination of quercetin (Q) and alantolactone (A) was found to induce synergistic immunogenic cell death (ICD) at molar ratio of 1:4 (Q:A). To achieve the ratiometric loading and delivery, the micellar delivery of Q and A (QA-M) was developed with high entrapment efficiency and drug loading at optimal ratio. QA-M achieved prolonged blood circulation and increased tumor accumulation for both drugs. More importantly, QA-M retained the desired drug ratio (molar ratio of Q to A=1:4) in tumors at 2 and 4 h after intravenous injection for synergetic immunotherapy. Tumor growth was significantly inhibited in murine orthotopic CRC by the treatment of QA-M, when compared to PBS and a combination of free drugs (p<0.005). The combination nano-therapy stimulated the host immune response to effectuate long-lived tumor destruction and induced memory tumor surveillance, with a 1.3-fold increment in survival median time when compared to PBS (p<0.0001) and a combination of free drugs (p<0.0005). The results of presently disclosed subject matter demonstrate the synergistic therapeutic effect induced by co-delivery of Q and A, which are capable of reactivating anti-tumor immunity by inducing ICD, causing cell toxicity, and modulating the immune-suppressive tumor microenvironment. Such a combination of Q and A with synergistic effects entrapped in a simple and safe nano-delivery system may provide the potential for scale-up manufacture and a novel clinical use as an immunotherapeutic agent for CRC.

### 1.2 Introduction

### 1.2.1. Background

Colorectal cancer (CRC) is a one of the leading causes of cancer death over the world that affects both women and men. It is the third major cause of death in US. American Cancer Society, 2017. Surgical resection, chemotherapy (CapeOX, FOLFOX, or FOLFIRI), and radiotherapy are standard clinical treatments. These regimens, however, are not effective for the advanced stage of the disease. High recurrence rate after surgery is still troublesome. Weitz et al., 2005; McKeown et al., 2014; and Birendra et al., 2017. In 2017, more than 95,520 new cases of colon cancer and about 40,000 new cases of rectal cancer were reported in the U.S. alone. Although colonoscopy and other screening and preventative measures improve the survival rates, less than 40% of CRC can be diagnosed at a localized stage. The five-year survival rate dramatically falls from 90% at the local stage to only 14% when the cancer metastasis occurs, for example, in the liver. American Cancer Society, 2017; Goodwin and Huang, 2017.

Recently, effective immunotherapies have come to the clinical reality and the forefront of treatment regimens for cancer. Cancer immunotherapy is thought to strengthen immune responses by either stimulating activities of the immune cells or blocking signals produced by cancerous cells to suppress immune responses. Alev et al., 2018. It has been confirmed that, in a tumor microenvironment, immune cells could regulate tumor progress and are attractive therapeutic targets. Gajewski et al., 2013; Wellenstein and de Visser, 2018; and Duan 2018. In recent years, treatment approaches based on modulating the immune system were successful in treating a variety of cancers. These approaches include immune blockade inhibitors that interfere with the programmed death-1/programmed death-ligand 1 (PD-1/PD-L1) to overcome immune suppression or chimeric antigen receptor T-cell therapy by engineering patient T-cells to recognize and attack cancer cells. Marin-Acevedo et al., 2018; Showalter et al., 2017. In CRC, patients with defective DNA mismatch repair system (MMR) or microsatellite instability (MSI-H) are more responsive to immunotherapy. Unfortunately, only 5%-15% of patients display MMR-deficient/MSI-H and checkpoint blockade immunotherapies could be very effective for patients whose tumors are pre-infiltrated by T cells. Goodwin and Huang, 2017; Pfirschke et al., 2016. Unfortunately for colorectal cancer patients, about 95% of the patient population does not respond to the PD-1/PD-L1 blockade treatment. Song et al., 2018; Gilabert-Oriol et al., 2018.

It was reported that some chemotherapeutic drugs (e.g., mitoxantrone, doxorubicin, bortezomib, oxaliplatin, paclitaxel, and gemcitabine) exhibit immune-modulating effect. These drugs could potentially be harnessed for clinical enhancement of tumor-specific immunity and modulate outcome of malignant diseases. Hodge et al., 2013; Suryadevara, et al., 2017; Kono et al., 2013; and Zhang et al., 2018. These agents could induce immunogenic cell death (ICD), act like to transfer tumor cells into “therapeutic vaccines” or directly stimulate the immune response through either promoting maturation and activation of immune cells, or inhibiting immunosuppression of immune cells, such as myeloid-derived suppressor cells (MDSCs) and T regulatory cells (Tregs). Gubin and Schreiber, 2015; Tesniere et al., 2016; and Obeid et al., 2007. ICD is characterized by the expression of calreticulin (CRT) on the membrane of dying tumor cells, providing an “eat-me” signal for the uptake by dendritic cells (DCs). Obeid et al., 2007; Martins et al., 2014. The following release of adenosine triphosphate (ATP) and high-mobility group box 1 (HMGB1) protein from the tumor cells acts like the adjuvant stimuli to the antigen presenting DC. Kroemer et al., 2013; Lu et al., 2017. Therefore, induction of ICD has become a novel immunogenic treatment to control aggressive, metastatic, or recurrent cancers. Gubin and Schreiber, 2015. The clinical advantages and limitations of conventional cytotoxic chemical drugs are obvious, however, especially in the destruction of the immune system. Crawford et al., 2004. The combination of mitoxantrone and celastrol derived from root of the classic Chinese medicinal herb to trigger ICD and elicit systemic immunity has been previously reported. Liu et al., 2018. The clinic use of celastrol, however, is limited by its narrow therapeutic dose window and adverse effects, such as infertility and cardiotoxicity. Wang et al., 2011; Cascao et al., 2017.

### 1.2.1. Scope of Work

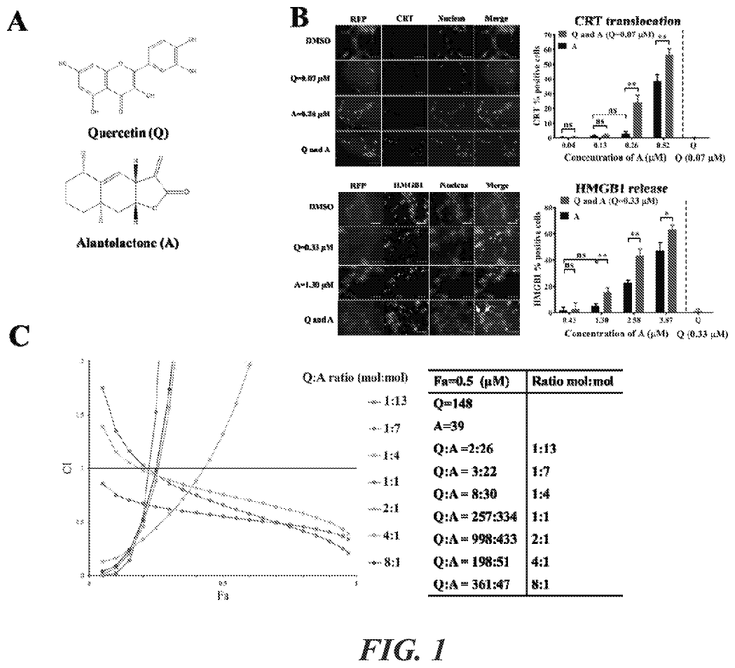

In the presently disclosed subject matter, other traditional Chinese medicines were screened and it was unexpectedly found that quercetin (Q) could work in synergy with alantolactone (A) to induce ICD. The chemical structures of quercetin (Q) and alantolactone (A) are shown in FIG. 1A.

Q as a member of the bioflavonoid family and exhibits a wide spectrum of beneficial effects, such as anti-inflammatory, antioxidant, antiproliferation, and anticancer activities and metastasis. Ward et al., 2018a; Feng et al., 2018; Rockenbach et al., 2013. It has attracted abundant interest because of its therapeutic properties, along with its safety profile (GRAS-generally recognized as safe report) and natural origin (it is extensively distributed in daily diet including green vegetables, onions, berries, and so forth). Egert et al., 2008. The anti-cancer effects of Q were reported in several cancers, such as pancreatic, breast, cervical, and prostate cancers. Ward et al., 2018a; Rockenbach et al., 2013; Ward et al, 2018b.

A is a major bioactive sesquiterpene lactone component of Inula racemosa Hook.f. and it is attributed with several beneficial activities, including anti-bacterial, anti-inflammatory, and anti-tumor activities through the mechanism of apoptosis. Chun et al., 2012; Rasul et al., 2013. Its targeting apoptosis arises from suppression of activated signal transducer and activator of transcription 3 (STATS) and induction of overloaded reactive oxygen species (ROS) causing massive oxidative deoxyribonucleic acid damage, glutathione depletion, and mitochondrial dysfunction, which eventually lead to apoptosis. Chun et al., 2015; Khan et al., 2012.

Little information exists, however, concerning the role of Q and A in antitumor immunity and tumor progression in immunosuppressive tumor environment. A combination of Q and A was prepared and their synergy in triggering ICD and inducing cell apoptosis was confirmed on CT26-FL3, a murine model of microsatellite stable CRC that has been inoculated in the wall of the colon as an orthotopic model. To maintain the optimal molar ratio of Q and A not only in the process of drug loading, but also in tumor tissue after injection, long-circulated micellar particles were employed to co-deliver Q and A (QA-M), taking the hydrophobic properties of both drugs into consideration. This co-delivery of Q and A in micelles is assumed to prime robust innate and adaptive immune responses, induce cancer cells apoptosis and control cancer progression, which would elicit prolonged survival of the host.

### 1.3 Results and Discussion

### 1.3.1 Evaluation on the Synergistic Effect of Q and A on Inducing ICD and Cell Apoptosis

Currently, increasing efforts are focusing on the application of certain stress agents that can induce ICD in cancer cells. Kawano et al., 2016. The immunogenic characteristics of ICD are mediated mainly by damage-associated molecular patterns, which include cellular surface-exposed CRT and release of HMGB1. Q was reported to evoke ER stress via up-regulating glucose-regulated protein 78 and C/-EBP homologous protein as markers of ER stress and leading to the cleavage of caspase-4, which is an ER-resident caspase. Liu et al., 2017. Little research about the ICD effect of either Q or A has been reported.

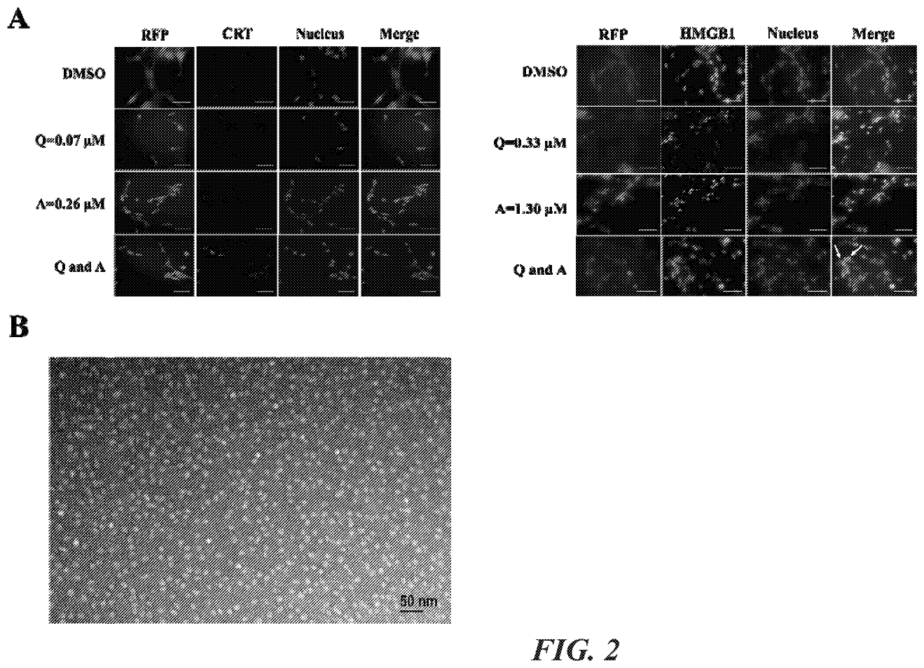

In the presently disclosed subject matter, the ICD effect was studied by using immunofluorescence. As shown in FIG. 1B (enlarged pictures of each group can be found in FIG. 2A), after incubation with different concentrations of free Q, at the concentration of 0.07 and 0.33 μM, it was affirmed that Q exhibited minimum or undetectable effect on CRT translocation and HMGB1 release, respectively. On the other hand, A alone induced a concentration dependent ICD effect in both CRT translocation and HMGB1 release. Both effects could be enhanced by combining with Q (0.07 and 0.33 μM for CRT translocation and HMGB1 release, respectively). As for CRT translocation, with the lower concentrations of A at 0.04 or 0.13 μM, both the A and QA exhibited little difference when compared to control group (DMSO-treated group). When 0.26 μM of A was incubated with 0.07 μM of Q on CT26-FL3 cells for 4 h, however, a 2.1-fold increment was observed on % CRT-positive cells when compared with A alone (p<0.0005). With the increased concentrations of A, the QA combination showed more obvious translocation of CRT. The same trend was observed in the release behavior of HMGB1. Both Q and A at the concentration of 0.33 μM and 1.3 μM, separately, showed undetectable release of HMGB1, as compared to control group. HMGB1 positive cells, however, were increased by the combination of these two drugs at these concentrations. Thus, these results confirm that Q and A work synergistically to induce ICD at low concentrations.

In addition to the ICD effect arising from the application of QA on CT26-FL3 cells, the cytotoxicity caused by QA combination was further investigated. Q induces apoptotic and necrotic cell death of malignant cells without effecting normal epithelial cells, which relates to its effect on modulating ROS production and interfering with Akt and NF-κB signaling pathways. Ward et al., 2018b. In vitro cytotoxicity analysis of the QA combination on CT26-FL3 cells after 24 h incubation (FIG. 1C) was investigated. The combination index (CI) vs fraction of the affected cells (Fa) was plotted with different molar ratios of Q and A. CI values below 1 indicate synergy. Miao et al., 2014. When Q and A were combined, with the drug ratios shown, the CI values below 1 were found at molar ratios of 1:13, 1:7 and 1:4 (Q:A mol/mol). Significantly lower IC₅₀ for Q was found in the QA combination (IC₅₀=8.0 μM), which was 94.8% less than that of Q alone (IC₅₀=148 μM). It indicates that A increased the sensitivity of CT26-FL3 cells to Q during incubation. With the consideration of the synergistic effect of QA at ICD and cytotoxicity, the molar ratio of Q:A at 1:4 was selected for further in vivo experiments. Next, Q and A-loaded micelles (QA-M) were prepared and their synergistic effect in vivo was investigated.

### 1.2.2 Preparation and Characterization of QA-M

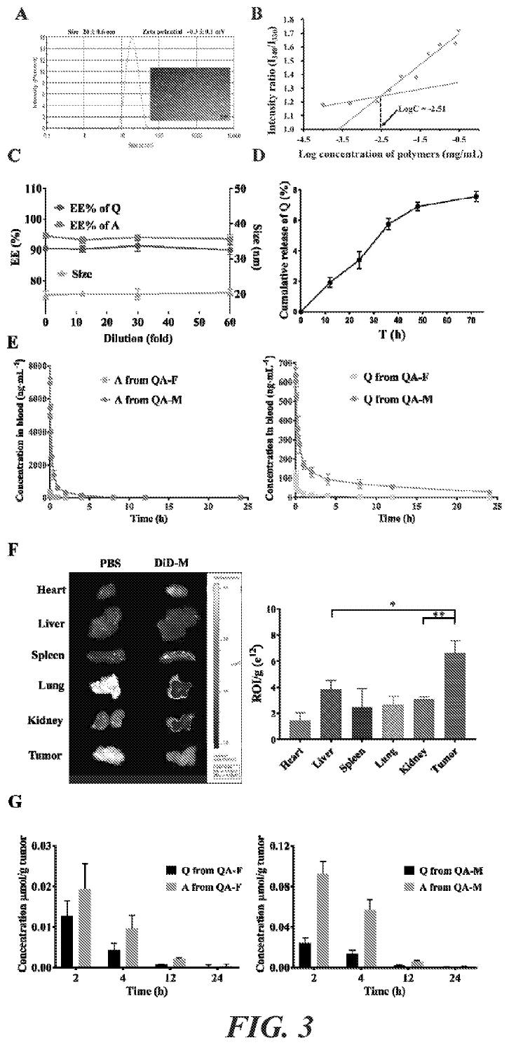

QA-M were prepared with 1,2-distearoyl-sn-glycero-3-phosphoethanolamine-N-methoxy-poly(ethylene glycol 2000) (DSPE-PEG2000) and D-α-Tocopherol polyethylene glycol succinate (TPGS) by the ethanol injection method. The morphology of QA-M is shown in FIG. 3A (enlarged TEM photo of QA-M can be found in FIG. 2B). Particles were spherical with a narrow size distribution at 20±0.6 nm and zeta potential at −0.3±0.1 mV. The encapsulation efficiency of QA-M was 90.5±0.6% for Q and 94.6±0.8% for A (molar ratio for encapsulated Q to A is 1:4), respectively. The drug loading of QA-M was calculated as 0.90±0.01% for Q and 2.80±0.02% for A. Thus, the ratiometric loading of Q and A was achieved in these micelles.

The critical micelle concentration (CMC) value indicates the stability of micelles. Micelles disassemble at concentrations below the CMC, while the polymer aggregates and form micelles at concentrations above the CMC. The lower the CMC value of a polymer in preparation, the more predicted stability of the micelle particles. Jin et al., 2018. The CMC of DSPE-PEG2000 and TPGS were reported to be 0.0336 mg/mL and 0.2 mg/mL, respectively. Sezgin et al., 2006; Mi et al., 2011. In FIG. 3B, the CMC of mixed micelles prepared by the ethanol injection method was much lower than that of either DSPE-PEG2000 or TPGS alone. The rather low CMC at 0.0031 mg/mL of QA-M suggests its predicted antidilution stability in systemic blood circulation. This observation is likely the result of the enhanced hydrophobic interaction between the hydrophobic blocks of DSPE-PEG2000 and TPGS. Jin et al., 2018.

To further prove the stability of QA-M, the dilution stability was investigated. The micelle systems were diluted 12-, 30- and 60-fold in PBS buffer (pH=7.4). The size distribution and drug entrapment efficiency were recorded for 24 h at 37° C. (FIG. 3C). When diluted to 60-fold, the concentration of QA-M was still above the CMC, therefore the micelles would not dissociate and there is no significant change found in size distribution and entrapment efficiency before and after the dilution.

The release behaviors of Q and A from QA-M are shown in FIG. 2D. The release percentage of Q to total Q from QA-M was only (7.6±0.3)% even after 72 h. As for the release of A from QA-M, it was not detectable throughout the experiment (the lowest detection limit for Q: 100 ng/mL, equals to 0.3% of Q in QA-M; the lowest detection limit for A: 100 ng/mL, equals to 0.1% of A in QA-M), which indicated that a small amount of A was released under the sink condition formed by the high concentration of soft liposomes (lecithin concentration 100 mg/mL). The controlled release of Q and A from QA-M shows that: (1) the release medium would not sabotage the structure of QA-M, not like surfactants. To be distributed into soft liposomes, the loading drugs need to dissolve in water in molecular form at the very beginning. Because of the higher hydrophobicity of A than that of Q, the less trend for A to be transferred to lecithin vesicles (the concentration of A in the release medium was under the lowest detection limit), while Q released from micelles for about 7.6% at 72 h; (2) the sustained release of both Q and A makes it possible for the micelles to maintain optimal drug ratio in vivo. In fact, together with the dilution stability and in vitro release results, it was assumed that the QA-M would stay stable under the dilution of blood once injected in the vein and keep the drugs entrapped at an almost unchanged ratio during the blood circulation time. The following pharmacokinetics and tissue distribution study were conducted to confirm this assumption.

Due to the stability within the blood stream and the particle membrane modification of polyethylene glycol, prolonged circulation of QA-M within the body would be predicted and QA-M would effectively deliver the cargo to the tumor through the enhanced permeability and retention (EPR) effect. Jin et al., 2018; Sezgin et al., 2006. Thus, the pharmacokinetics and biodistribution of QA-M were investigated. As shown in FIG. 3E, the micellar drugs exhibited prolonged circulation in the blood stream for both Q and A, compared to free combination of Q and A (QA-F), calculated by a using non-compartment model with PKsolver (Table 1). Q and A of QA-M particularly exhibiting 15.7-fold and 16.3-fold higher in the area under the concentration-time curve from zero to the final time point (AUC₀₋ₜ) than Q and A of QA-F, respectively.

The apparent volumes of distribution during the terminal phase (V_{z}) of Q and A of QA-M were significantly decreased, which were 13.3% and 14.2% of the Q and A of QA-F, separately. The results showed that the micellar nanodrug could prolong the circulation time and slow down the drug distribution. The near-zero zeta potential and the presence of polyethylene glycol in QA-M are likely responsible for the long-circulating effect of micelles. Mi et al., 2011; Parveen et al., 2011.

TABLE 1
Pharmacokinetic parameters of QA-F and QA-M (n = 6).

| | Q from QA-F | Q from QA-M | A from QA-F | A from QA-M |
|---|---|---|---|---|
| MRT_{0→t} ¹(h) | 2.84 ± 0.61 | 14.71 ± 2.32* | 4.33 ± 0.98 | 7.90 ± 1.15§ |
| AUC_{0→t} ²(ng/mL · h) | 105.78 ± 8.21 | 1767.98 ± 109.78* | 223.02 ± 13.42 | 3878.67 ± 299.86§ |
| V_{z} ³(L) | 172.79 ± 10.32 | 23.01 ± 3.65* | 307.34 ± 49.23 | 43.90 ± 9.77§ |
| CL ⁴(L/h) | 47.23 ± 8.43 | 1.33 ± 0.25* | 48.61 ± 5.97 | 2.06 ± 1.03§ |

¹The in-vivo residence time from time zero to the final time point;
²The area under the concentration-time curve from zero to the final time point;
³The apparent volume of distribution;
⁴The total plasma clearance.
*vs Q from QA-F, p < 0.05;
§vs A from QA-F, p < 0.05.

DiD (1,1′-dioctadecyl-3,3,3′,3′-tetramethylindodicarbocyanine, 4-chlorobenzenesulfonate salt) was used as a probe for micelle distribution in CT26-FL3 tumor-bearing mice. DiD-loaded micelles (DiD-M) were detected mainly in the tumors at 24 h after injection (FIG. 3F). Even though a certain amount of micelle accumulated in the liver or lungs, with the help of PEGylated micelles, most of the micelles accumulated in the tumor. At least a 1.7-fold increment of relative fluorescence intensity was found in DiD-M group when compared with other major organs. Furthermore, biodistribution of Q and A was detected after i.v. injection of QA-M or QA-F at the dose of 3 mg/kg of Q and 9 mg/kg of A, respectively (FIG. 3G). After entrapped in micelles, both Q and A could exhibit at least around 2-fold and 5-fold increment accumulation in tumor when compared to Q and A from QA-F within 4 h, respectively. The optimal ratio of Q and A obtained from ICD effect in vitro was realized with the concentration of drugs in tumor at early time point. The ratio of Q and A after delivered by micelle, was 1.0:3.8 and 1.0:4.1 at 2 and 4 h after injection, respectively, which was approximately the same as the optimal molar ratio at 1:4 obtained from ICD effect in vitro. At the other two measured time points, 12 and 24 h, content of both Q and A in tumors were decreased too dramatically to retain the optimal ratio because of metabolism in vivo. The QA-F failed, however, to deliver Q and A to reach such a ratio (1.0:1.6, 1.0:2.3, 1.0:3.0 and 1.0:1.6 at 2, 4, 12 and 24 h, respectively). With the pharmacokinetic and biodistribution profiles of QA-M, it is thought that the benefit of using a micellar drug delivery system is not just to prolong the blood circulation and increase the tumor accumulation, but also the co-delivery of Q and A at the optimal molar ratio to the tumor for synergistic ICD and cytotoxicity. Thus, the micelles enabled ratiometric loading, as well as ratiometric delivery of Q and A. Accordingly, synergistic drug action was expected.

### 1.3.3 Therapeutic Efficacy in Orthotopic Colorectal Cancer Model

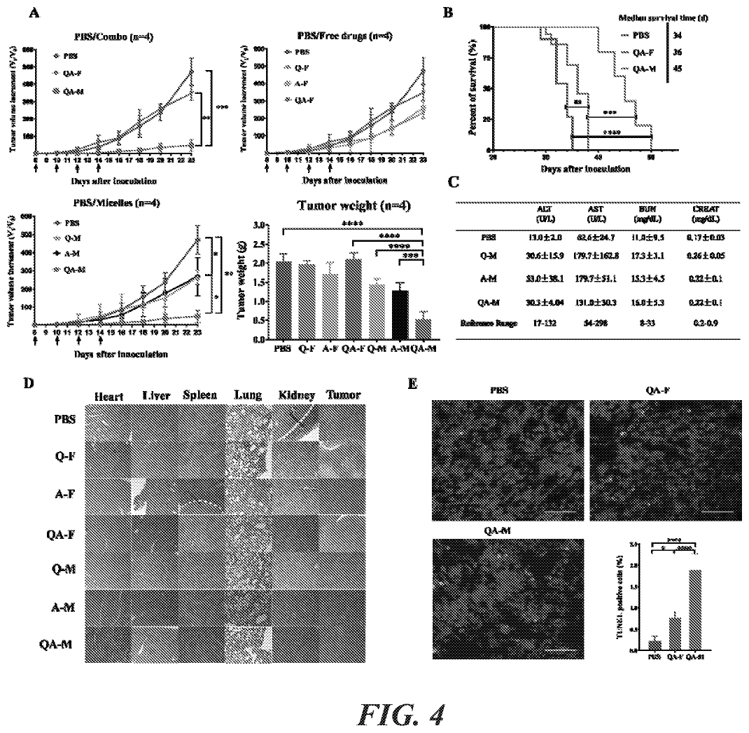

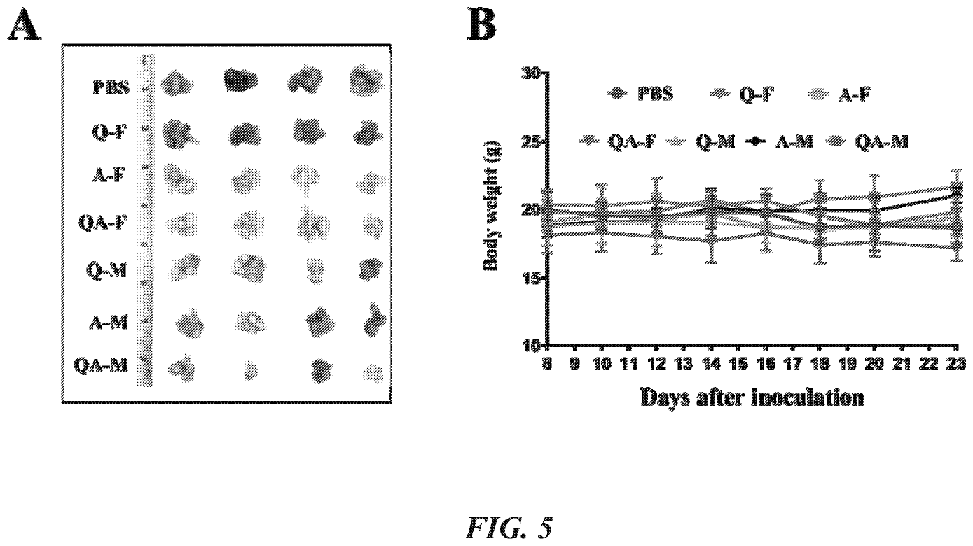

To demonstrate the utility of QA-M for immunotherapy against colorectal cancer in vivo, their inhibition on the growth of orthotopic CT26-FL3 tumors was investigated. These tumors lack T-cell infiltration, Gilabert-Oriol, et al., 2018, and therefore are resistant to conventional immunotherapy. CT26-FL3 tumor-bearing mice were administered with free or micellar Q and A combinations four times every other day (FIG. 4A, detailed data of other groups can be found in FIG. 5A). QA-M combination therapy significantly delayed tumor growth (p<0.0005), and the increment of tumor volume of this group was approximately 10% of that of the group treated with PBS, while QA-F had no impact on tumor progression. The Q-M or A-M alone also could show tumor growth inhibition to a certain degree. But after the treatment was terminated, tumor growth resumed. Importantly, QA-M did not cause any body weight change, in contrast to PBS groups and free drugs-treated groups, which showed some weight decline at the late stage of tumor progression (FIG. 5B). QA-M also showed significantly prolonged median survival time, when compared to PBS and QA-F groups (p<0.0001) (FIG. 4B). The levels of alanine aminotransferase (ALT), aspartate aminotransferase (AST), blood urea nitrogen (BUN) and creatinine in Q-M, A-M and QA-M groups also were in the normal range, indicating the absence of liver and kidney toxicities (FIG. 4C). Zhang et al., 2013. Metastasis in liver and spleen was observed in H&E staining of the PBS and A-F treated groups, respectively. Q-M, A-M and QA-M groups, however, did not show obvious kidney injury, pulmonary toxicity, cardiac damage, or inflammatory infiltrates in the spleen (FIG. 4D).

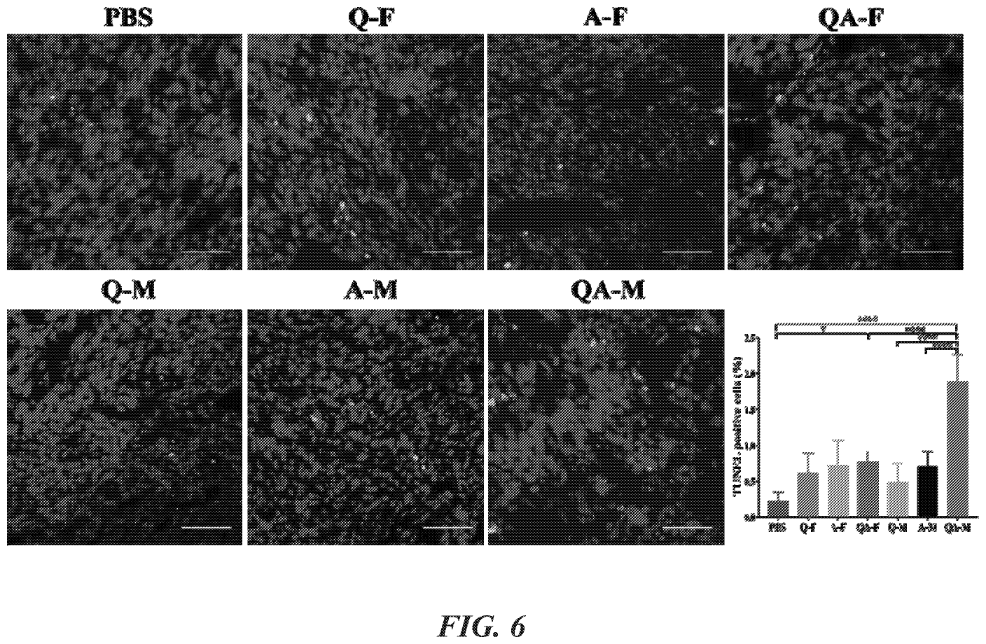

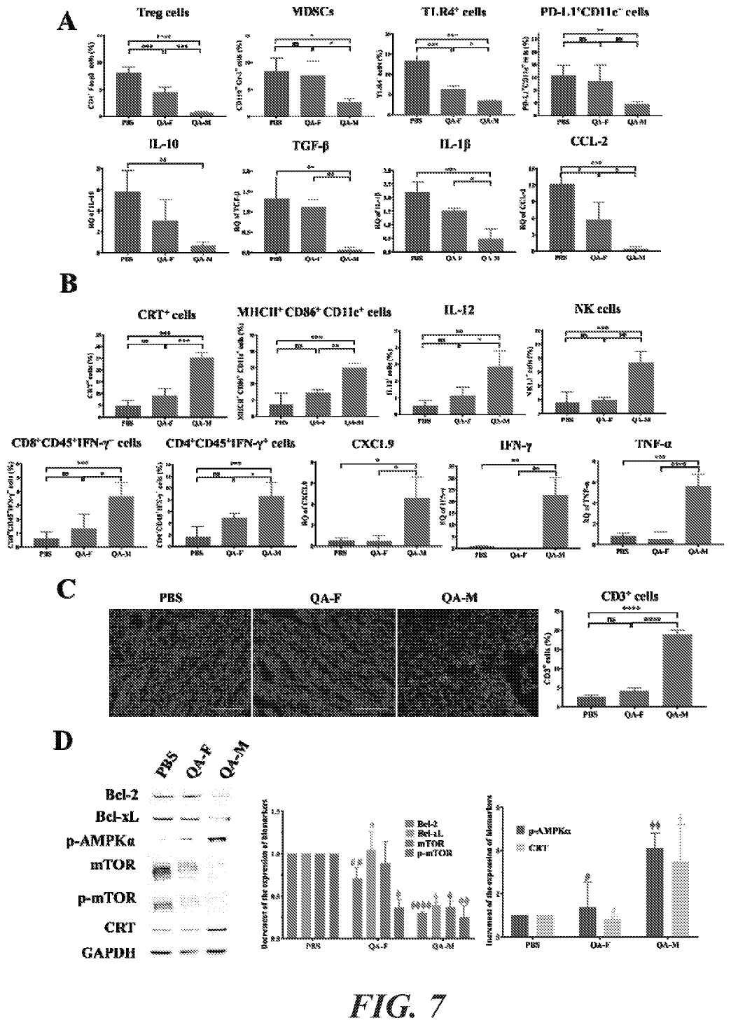

Tumors were collected at the end of the experiment and analyzed for effective apoptosis, immunosurveillance, and other mediators of antitumor immune stimulation. Since the activity of Q-F and A-F was very similar to that of QA-F (FIG. 4A), only QA-F was chosen for detailed analysis. Firstly, the TUNEL assay (FIG. 4E, detailed data of other groups can be found in FIG. 6) revealed that QA-M exhibited the most effective killing effects, and to induce a 6.6-fold and 2.0-fold as high as the number of apoptotic cells compared with the control group and QA-F treated group, respectively. Significant characteristic of cancer cells is the loss of regulation on cell cycle, which allows continuous proliferation. Q arrests the cell cycle in G2/M phase, while A induces cell cycle G1/G0 phase arrest. Lee et al., 2006; Zhao et al., 2015. Thus, both drugs exhibit antiproliferative roles on cancer cells, besides inducing apoptosis. As it also can be found in FIG. 7D that QA-M caused significant reduction in the expression of B-cell lymphoma 2 (Bcl-2) and B-cell lymphoma-extra large (Bcl-xL) proteins, which caused a 57.2% and 70% reduction of expression compared with the QA-F and control group, respectively. This finding is consistent with data in tumor growth inhibition, showing that the QA-M exerted greater antitumor effects than the free drug combination.

1.3.4 Tumor Immune-Microenvironment Changes after Various Treatments

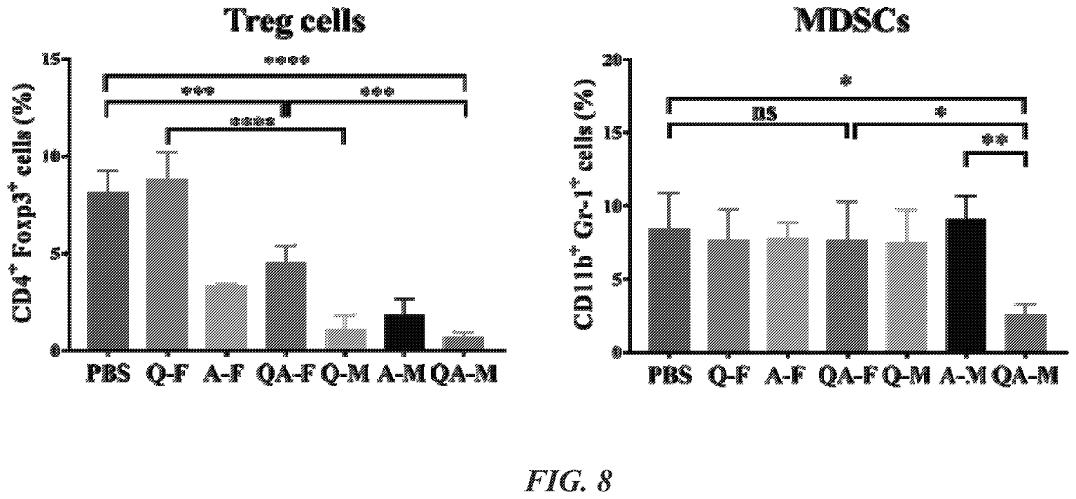

Tumors also are known to employ several immunosuppressive mechanisms to prevent antitumor immune responses. Wu et al., 2018; Fridman et al., 2012. QA-M could significantly reduce the immunosuppressive cell populations (FIG. 7A, detailed data of other groups can be found in FIG. 8). Significant reduction of the strongly immunosuppressive Tregs and MDSCs was observed. Tumor content of Tregs (CD4⁺Foxp3⁺ T cells), which has been correlated with the poor prognosis of cancer patients, Sakaguchi et al., 2010, was reduced by 91.1% in QA-M group compared to the untreated control (p<0.0001). QA-M also reduced the percentage of MDSCs from 8.4±2.4% in the untreated group and 7.6±2.6% in QA-F group to 2.6±0.6% in QA-M group, reaching statistical significance (p<0.05). Accumulation of MDSC in tumor microenvironment promoted tumor cell survival and suppressed proliferation and functional activity of T cells. Xu et al., 2013.

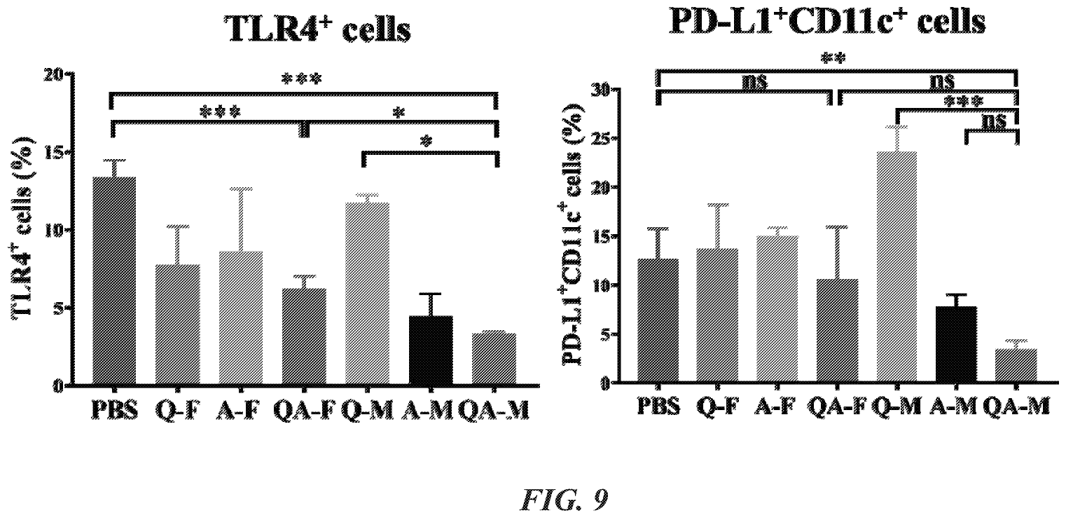

The treatment also can inhibit tumor-promoting inflammation (FIG. 7A, detailed data of other groups can be found in FIG. 9). Toll-like receptor 4 positive (TLR4⁺) cells were analyzed and both QA-F and QA-M decreased the percentage from 13.4±1.1% in PBS group to 6.3±0.8% in QA-F group and 3.4±0.1% in QA-M group. Over-expression of TLR4 in CRC is associated with immune suppression and resistance to therapy. Li et al., 2014; Yesudhas et al., 2014. Inflammation is central to the development of cancer. Anti-inflammatory drugs can increase the efficacy to treat CRC. Wang and DuBois, 2013. The anti-inflammatory role of Q mainly results from its inhibitory effect on pro-inflammatory cytokines tumor necrosis factor alpha (TNF-α), interleukin 6 (IL-6), and interleukin-1β (IL-1β), and inflammatory mediators, such as catalase and nitric oxide. Li et al., 2016. A was used clinically as anti-inflammatory agents as described in the China Pharmacopoeia and European Pharmacopoeia by inhibiting the expression of cyclooxygenase 2 and attenuating the binding between cyclooxygenase 2 and NF-κB cells. Wang et al., 2013. PD-L1 expression on CD11c positive cells in QA-M also was observed to be significantly reduced, which is 27.5% of that in PBS group. PD-L1 is expressed on the cell surface of activated antigen-presenting cells and select tumor cells that constrain immune responses. It was demonstrated that PD-L1 expressed on dendritic cells (DCs) inhibits naive and effector T cells. Sage et al., 2018. The decreased expression of PD-L1 on DCs of QA-M group could improve the activity of anti-tumor T-cell response. The decreased secretion of immune-suppressive cytokines IL-10, TGF-β, IL-1β, and CCL2 also were found in QA-M group. Thus, QA-M has exerted a strong anti-inflammatory effect in the treated tumor.

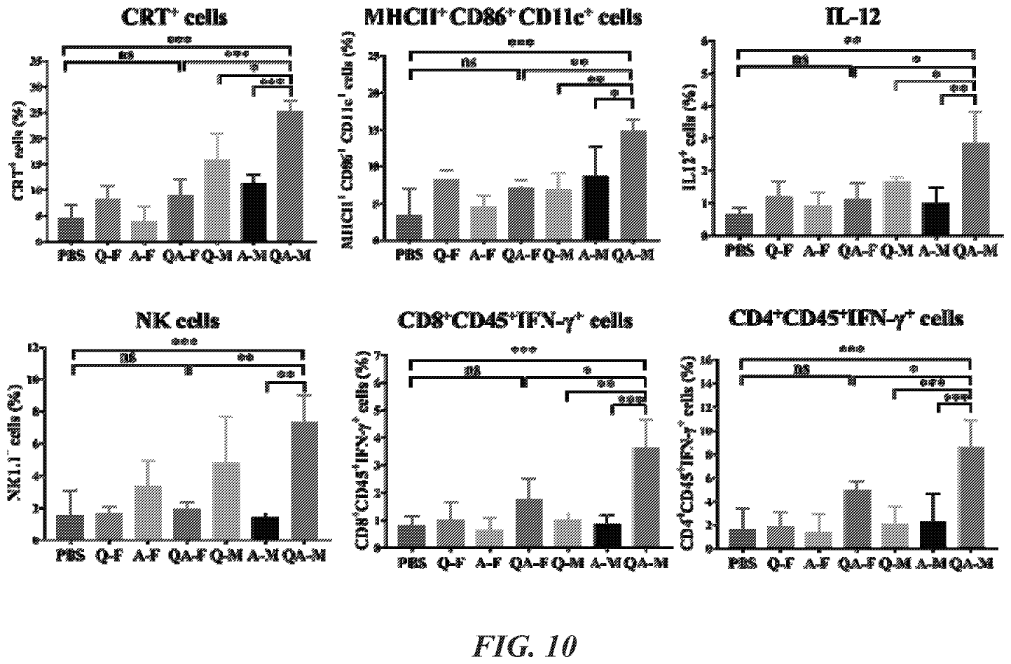

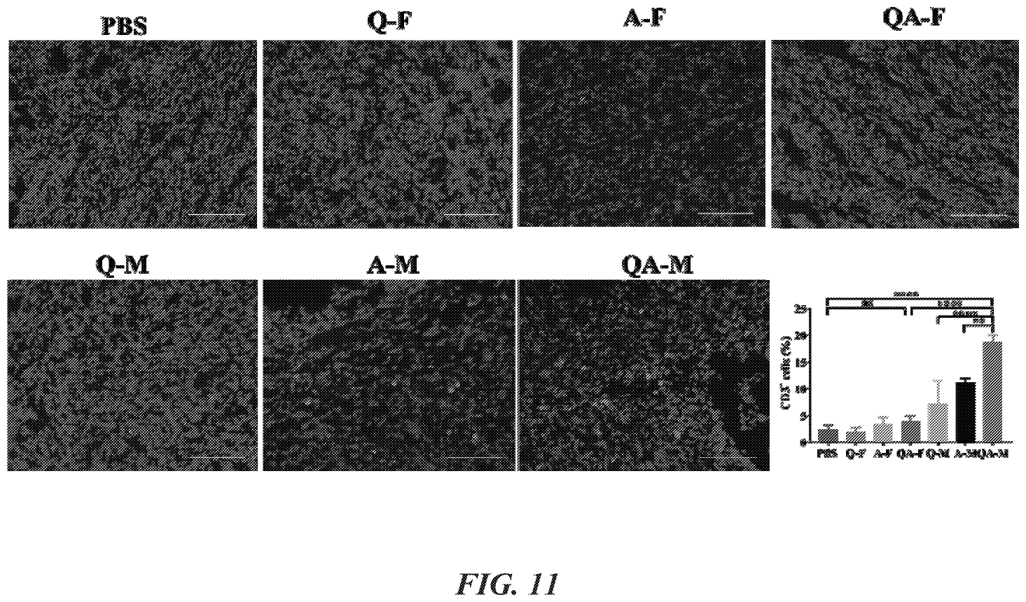

There was a stringent immune suppressive environment in the orthotopic colorectal tumor. Whether tumor-infiltrating immune cells in the tumor could be appropriately activated was investigated. Importantly, a dramatic increment in CRT⁺ cells were observed in QA-M-treated group, which was in accordance to the ICD effect in FIG. 7B. The synergetic effect of ICD induced by co-delivery of Q and A inside the orthotopic CRC tumor strongly reactivates anti-tumor immunity. ICD released HMGB-1 and CRT, which in turn activated DCs. QA-M exhibited the greatest effect on CD3⁺ T cell, CD8⁺ and CD4⁺ T cells in the tumor, which was increased 7.4-, 4.4-, and 6.8-fold as compared to the control group, respectively (FIG. 7B and FIG. 7C, detailed data of other groups can be found in FIG. 10 and FIG. 11). It is known that maturation of DCs is associated with increased expression of MHC class II and co-stimulatory molecules, such as CD40, CD80 and CD86 on the cell surface. Palucka et al., 2012. The presently disclosed results showed that QA-M treatment greatly increased the levels of costimulatory signal (MHC class II and CD86) on dendritic cells, suggesting that these DCs are matured and activated to promote antitumor T cell response and induce cytokine secretion, such as interleukin 12 (IL-12). Flow cytometric analyses showed that the lymphoid cell population for natural killer (NK) cells was significantly and positively affected by QA-M. IFN-γ production from CD4⁺ and CD8⁺ T cells in tumors was significantly increased after QA-M treatment. It demonstrated that T cells were activated, which was likely the major reason for the prolonged survival time of the host. Interferon-γ (IFN-γ) is the Th1 cytokine and is critical for the development of cell-mediated antitumor immune responses. QA-F did not increase IL-12 and IFN-γ as compared with the untreated group, while QA-M treatments induced significantly higher levels of IL-12 and IFN-γ in the tumor. C-X-C motif chemokine 9 (CXCL9) is one of the cytokines produced in response to interferon-γ (IFN-γ) and triggers inflammation with the accumulation of activated lymphocytes. Han et al., 2017. QA-M significantly increased the expression of CXCL9. Interferons not only exhibit important antiviral effects, but also exert a key influence on the quality of the cellular immune response and amplify antigen presentation to specific T cells. Le et al., 2000. Compared with the control group and QA-F, QA-M also could increase the secretion of tumor necrosis factor alpha (TFN-α) significantly. Thus, there was a significant down-regulation of the suppressive immune cells and cytokines with a concomitant up-regulation of immuno-active cells and cytokines by micellar co-delivery of Q and A to the tumor.

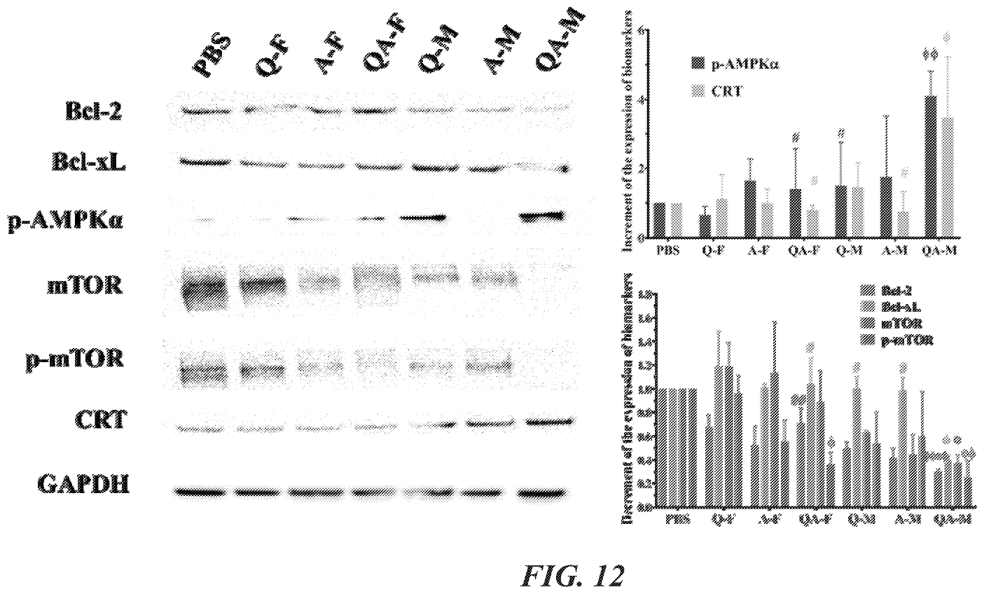

The western blot analysis of tumor lysates is shown in FIG. 7D (detailed data of other groups can be found in FIG. 12). When compared with the PBS group and the QA-F group, QA-M exhibited a significant increment in the expression of phosphor-AMP-activated protein kinase α (p-AMPKα) protein. For mammalian target of rapamycin (mTOR) and phospho-mTOR (p-mTOR), QA-M decreased the expression of both proteins. Autophagy is defined as the process by which cellular components are delivered to the lysosome and degraded to maintain essential activity and viability. Wang et al., 2018. The results indicated that QA-M could inhibit Bcl-2 to induce cell apoptosis and then promote the occurrence of autophagy. The other line of QA-M triggers autophagy is through producing p-AMPK and suppressing mTOR and p-mTOR. The protein kinase B (Akt)/adenosine monophosphate protein kinase (AMPK)/mTOR pathway is a key signaling link for metabolic pathways coordination and thus the nutrient supply balance. Kim et al., 2011; Kim et al., 2016. Q was reported to activate AMPK, an endogenous inhibitor of mTOR, by inhibiting mitochondrial ATP production through targeting and inactivating the mitochondrial F1F0-ATPase/ATP synthase and elevating AMP levels. Rivera et al., 2016; Ahn et al., 2008; Zheng and Ramirez, 2000. Like what was observed by flow cytometric detection, a significant 2.4-fold higher CRT level than that of PBS group also was observed.

### 1.3.5 Long-Term Anti-Tumor Immune-Memory Effects of QA-M

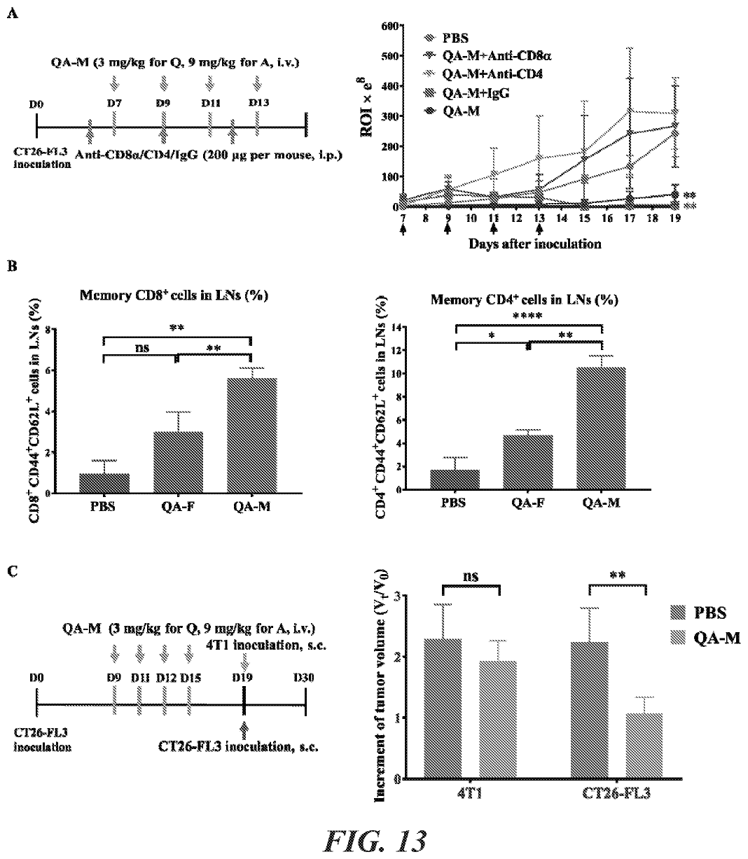

When CD4⁺ or CD8⁺ T cells in tumor-bearing mice were depleted before the treatment of QA-M, Song et al., 2018, the halted tumor growth disappeared by treatment of either anti-CD4 or anti-CD8α antibodies, while the isotype-matched IgG had no effect (FIG. 13A). The results suggested that immune surveillance T cells played an important role for the efficacy of QA-M. The activation of innate immune response in tumors of QA-M group induced the activation of adaptive immune response, therefore inhibiting tumor growth.

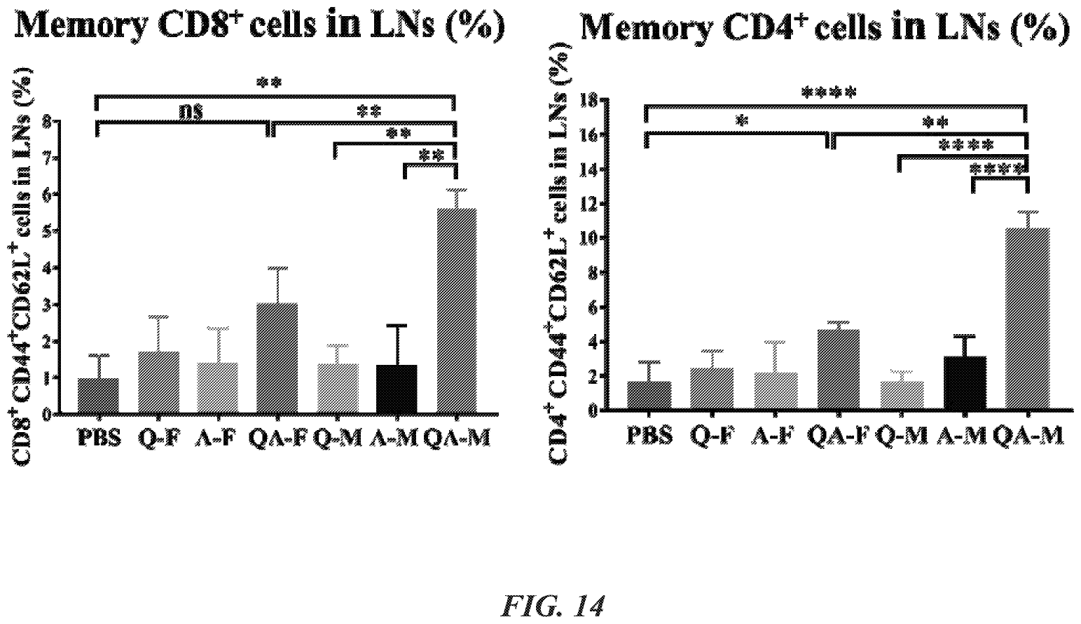

An important feature of immune memory response is its ability to induce a long-term memory response to antigenic challenge. In addition to the local cytotoxic T-lymphocytes (CTLs) being affected by the treatment of QA-M, another component of immune-surveilling cells, tumor-specific memory T cells, were dramatically recruited (FIG. 13B, detailed data of other groups can be found in FIG. 14). The increased memory CD8⁺ and CD4⁺ cells in lymph nodes (LNs) caused by QA-M, shows the effectiveness of ICD in vivo. Nineteen days after the inoculation of CT26-FL3 orthotopic colorectal tumor in mice, untreated group and treated group (four injections of QA-M every other day) received another challenge of CT26-FL3 cells and 4T1 cells subcutaneously at the same time. Eleven days later, tumors were measured (FIG. 13C). Tumor growth for 4T1 cells in both treated and untreated groups showed no significant difference. Tumor growth for CT26-FL3 in the treated group, however, was comparably slowed to about 50% of the untreated group. The result indicated a long-term memory response of the immune system was induced by QA-M which was specific for CT26-FL3 cells, but not for 4T1 cells.

### 1.3.6 Therapeutic Efficacy in Orthotopic Breast Cancer Model

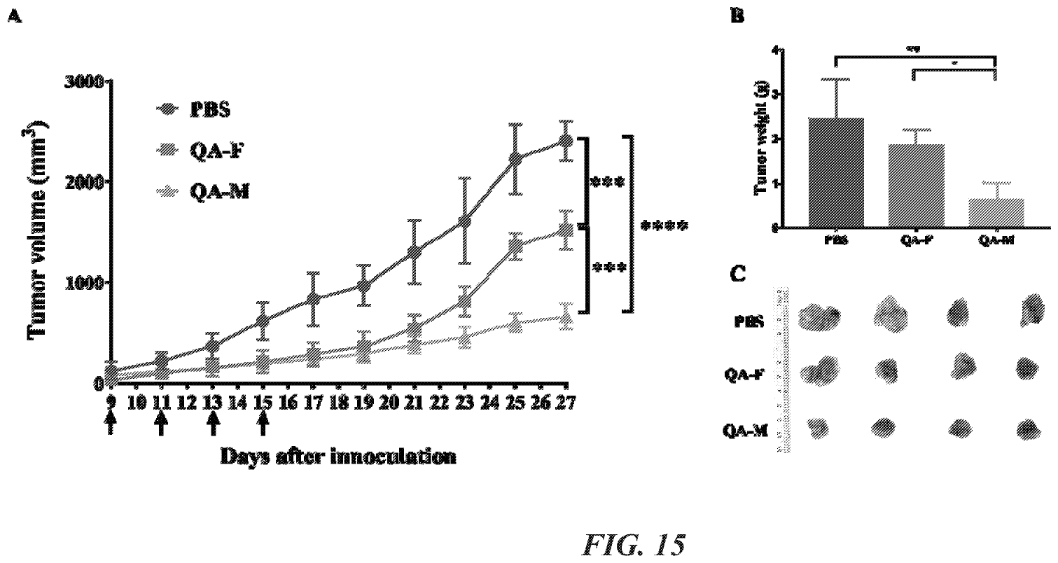

The triple negative breast cancer cells grown in the mammary fat pad of Balb/c mice also was examined for its response to QA-M. In PBS and QA-F treated groups, the continuous growth of tumor was observed. The group treated with QA-M showed a significant decrease in tumor growth rate (FIG. 15). The tumor weight of QA-M group was 26.1% and 34.2% of that of PBS and QA-F treated groups, respectively. These findings suggest that QA-M was effective in inhibiting tumor growth in 4T1 breast cancer.

### 1.3.7 Summary

Cancer cells have devised strategies to control cell death and limit the emission of danger signals from dying cells, thereby evading immunosurveillance. It was reported that tumors in around 95% of CRC patients are microsatellite stable, which are usually associated with fewer neoantigens and weak systemic immune stimulation. Goodwin and Huang, 2017. Here, Q at low concentration, at which no ICD effect by itself was observed, could help A to induce ICD effect characterized by CRT translocation and HMGB1 release. Furthermore, when combined with A at a certain ratio, Q could induce more cell death on CT26-FL3 cells, while the IC₅₀ of combined drugs was much lower than that of Q used alone. Therefore, QA-M was prepared with the aim to display the synergistic effect of ICD at an optimal molar ratio in vivo. Taking advantage of long-circulating and EPR effect resulting from the nanodrug delivery system, micellar suspension elevated the accumulation of Q and A in tumors and retained the optimal ratio at early time point after intravenous injection.

In addition to the ratiometric drug loading, thanks to the ratiometric biodistribution, a strong anti-tumor immunity was observed by the treatment of QA-M for the orthotopic CRC tumor and drastic anti-tumor growth effect in 4T1 breast tumor. ICD released HMGB1 and CRT can activate DCs for tumor antigen uptake and processing. Activated DCs are potent antigen presenting cells for a primary T lymphocyte response against tumor, the co-stimulatory signal (MHCII and CD86) on DCs upregulated can therefore successfully initiate anti-tumor T lymphocyte proliferation and cytokine secretion.

The balance between immune-effective cells, such as T cells, NK cells, and immunosuppressive cells—including Treg cells, M2 tumor associated macrophages, MDSCs—in the tumor microenvironment acts to calibrate the immune response to malignant cells. Major changes following this therapy included significant reduction of the strongly immunosuppressive Treg cells and MDSCs, inhibited tumor-promoting inflammation, greatly elevated expression of tumor infiltrating lymphocytes and chemokines and reduced autophagy. The tumor suppressive microenvironment was changed, while anti-tumor response and tumor surveillance were promoted. On a cellular level, it was demonstrated that the adaptive immune systems contribute to these systemic reactions and that NK cells also are increased. In addition, the release of danger signals or cytokines such as TNF-α and IFN-γ promoted DC maturation and cross-presentation, which resulted in the regression of more distant tumor masses through activation of tumor-specific T cells, since the increment of T cell in lymph note from tumor bearing mice were detected. After neutralizing of CD4⁺ and CD8⁺ T cells with monoclonal antibodies, the therapeutic effect was blocked. All these results demonstrated QA-M not only changed suppressive tumor microenvironment but also successfully promote systemic memory anti-tumor response.

Further, the formulation for QA-M was simply composed of two polymers, TPGS and DSPE-PEG2000. TPGS is used as safe adjuvant approved by U.S. Food and Drug Administration in Tocosol (Paclitaxel Nanoemulsion, Sonus Pharmaceuticals Incorporation) and DSPE-PEG2000 also is approved by the U.S. Food and Drug Administration as a component of anti-tumor product Doxil (Doxorubicin HCl Liposome Injection, ALZA Corporation). The safe and convenient protocol for the QA-M enables its potential for scale-up manufacture and clinical use as an immunotherapeutic agent.

### 1.4 Materials and Methods

### 1.4.1 Materials

Q (purity>95%), D-α-Tocopherol polyethylene glycol 1000 succinate (TPGS), puerarin and 1-naphthyl acetate and pyrene were purchased from Sigma-Aldrich (Sigma-Aldrich, Mo., USA). A (purity>98%) was purchased from Shanghai Tauto Biotech Co., Ltd. N-(Methoxypolyethylene oxycarbonyl)-1,2-distearoryl-sn-glycero-3-phosphoethanolamine (DSPE-PEG) was purchased from NOF Corporation (SUNBRIGHT® DSPE-020CN). DeadEnd™ Fluorometric TUNEL assay kits were obtained from Promega (Madison, Wis., USA). Antifade Mounting Medium with DAPI (4′,6-diamidino-2-phenylindole) was from Vector Laboratories (Burlingame, Calif., USA). DiD′ solid (1,1′-Dioctadecyl-3,3,3′,3′-Tetramethylindodicarbocyanine, 4-Chlorobenzenesulfonate Salt) was from Invitrogen (Carlsbad, Calif., USA). Egg yolk lecithin (PC-98T, PC>98%) was from Kewpie Corporation (Shibuya, Tokyo, Japan). All other chemicals were of analytical grade and were used as received.

### 1.4.2. Cell Lines

The original murine CT26-FL3 cells were kindly provided by Dr. Maria Pena at the University of South Carolina Murine and were transfected with vectors carrying RFP/Luc and puromycin resistance gene to express red fluorescent protein (RFP)/Luc. CT26-FL3 cells were cultivated in Dulbecco's Modified Eagle's Medium (DMEM, high glucose, Gibco) with 10% FBS and 1% penicillin/streptomycin (PS) (Invitrogen, Carlsbad, Calif.) at 37° C. and 5% CO₂. Murine breast cancer 4T1 cells were purchased from Tissue Culture Facility, UNC Lineberger Comprehensive Cancer Center and were cultivated in Roswell Park Memorial Institute (RPMI)-1640 medium with 10% FBS and 1% penicillin/streptomycin (PS) (Invitrogen, Carlsbad, Calif.) at 37° C. and 5% CO₂.

### 1.4.3 Animals

Six-week-old female Balb/c mice (20±2 g) were obtained from Charles River Laboratories. All animal handling procedures were approved by the University of North Carolina at Chapel Hill's Institutional Animal Care and Use Committee. Female Sprague-Dawley rats (200±20 g) were provided by Hunan SJA Laboratory Animals (Hunan, China). The animals were cared for in the animal experimental center at Jiangxi University of Traditional Chinese Medicine. The animal room was well ventilated and had a regular 12 h light-dark cycle throughout the experimental period.

### 1.4.4 Antibodies

InVivoMAb anti-mouse CD8α (Lyt 2.1), anti-mouse CD4 (clone GK1.5), rat IgG2b isotype were purchased from BioXcell (West Lebanon, N.H.).

1.4.5 Synergistic Effect on CRT Translocation and HMGB1 Release from the Cells

CT26-FL3 cells were seeded in 35-mm cell culture dishes with glass bottom at the density of 2×10⁵ per dish and incubated for 24 h before treatment. The cell culture medium was removed and replenished with different combination of Q and A containing media at the indicated concentrations for 4 h for CRT detection and for 8 h for HMGB1 detection. Cells were fixed and washed 3 times. A primary antibody, anti-CRT antibody (ab2907, 1:500, Abcam), diluted in cold blocking buffer (10% goat serum in PBS), was added for 60 min. After three washes in cold PBS, cells were then incubated for 60 min with the Alexa Fluor® 488 Goat Anti-Rabbit (IgG) (ab150077, 1:500, Abcam) diluted in a cold blocking buffer. Cells were fixed with 4% PFA for 20 min, and DAPI Mounting Medium was added for nuclear staining. For intracellular HMGB1 staining, cells were fixed and washed 3 times. Afterwards, cells were permeabilized with 0.1% Triton X-100 containing blocking buffer for 10 min, and rinsed three times with PBS, and nonspecific binding sites were blocked for 30 min A primary antibody for HMGB1 (ab79823, 1:500, Abcam) was added for 60 min. Cells were then incubated for 60 min with the Alexa Fluor® 488 Goat Anti-Rabbit (IgG) (ab150077, 1:500, Abcam) diluted in a cold blocking buffer. Cells were fixed with 4% PFA for 20 min, and DAPI Mounting Medium was added for nuclear staining. Slides were visualized under an Olympus IX81 inverted microscope under the 40× objective lens.

### 1.4.6 Synergistic Cytotoxicity Effect of Drugs Combination

The cytotoxicity of free Q, free A and drugs combination against CT26-FL3 cells were assessed using the MTT (3-[4,5-dimethylthiazol-2-yl]-2,5-diphenyltetrazolium bromide) method. Zhang et al., 2017. Cells were seeded in 96-well plates (5×10³ cells per well) and incubated for 24 h before treatment. Then the culture medium was withdrawn and fresh medium containing free drugs and drug combination with a series of concentration were added to each well. Following 24-h incubation, cell viability was determined by MTT assay. The synergy of Q and A was evaluated by using the Chou and Talalay method to calculate the combination index (CI). Zhang et al., 2014; Chou et al., 1984. CI was calculated by using the following equation (1):

CI=(D)₁/(Dₓ)₁+(D)₂/(Dₓ)₂ (1)

where (D)₁ and (D)₂ are the concentrations for single drug after combination that inhibit x % of cell growth, and (Dₓ)₁ and (Dₓ)₂ are the concentrations for single drug alone that inhibit x % of cell growth. CI values more than one or less than one demonstrate antagonism or synergism of drug combinations, respectively.

### 1.4.7 Preparation and Characterization of QA-M

QA-M were prepared with DSPE-PEG2000 and TPGS by the ethanol injection method. Briefly, Q and A (1:4, molar ratio) were first dissolved in ethanol used as a miscible solvent, together with carrier materials (1:6.5, molar ratio) including DSPE-PEG2000 and TPGS (1:4.8, molar ratio). Then the transparent organic solution was added dropwise to 2 mL of water at 60° C. under stirring for 30 min. The suspension was then dialyzed in distilled water for another 2 h at room temperature to remove the residual ethanol.

The preparation of Q-loaded and A-loaded micelles (Q-M and A-M) were by the same method as the QA-M except that Q or A was used alone. The preparation of DiD-loaded micelles (DiD-M, 47.5 μM) also was by the same protocol mentioned except that DiD was used to replace Q and A in the mixtures.

The mean size and zeta potential of micelles were measured by dynamic light scattering method using Malvern Zetasizer Nano-ZS90 (Malvern Instruments, Malvern, UK). All results were the mean of three test runs. The morphology of QA-M was observed under a JEM-1230 transmission electron microscope (TEM) (JEOL, Japan). Micelles were diluted with distilled water and negatively stained with phosphotungstic acid on a copper grid covered with nitrocellulose. Samples were dried at ambient temperature before observation.

The encapsulation efficiency (EE) of Q and A in micelles was calculated as the percent of the amount of drugs loaded in micelles over the original feeding amount. The drug loading content (DL) of micelles was calculated as the percentage of the amount of loaded drugs to the total amount of polymer used for loading. Briefly, 5 mg of free-dried QA-M was dissolved in 0.6 mL methanol and then the amount of Q or A in the solution were analyzed by high-performance liquid chromatography (HPLC) on a ZORBAX SB-C18 column (250 mm×4.6 mm², 5 μm; Agilent Technologies, Santa Clara, Calif., USA) at 268 nm. The mobile phase was 0.06 M ammonium acetate solution (pH 5.7, adjusted using glacial acetic acid)-acetonitrile. A gradient elution was used with a flow rate of 1.0 mL/min where initially 5% organic solvents (acetonitrile containing ammonium acetate solution) was held for 7 min, then increased linearly to 70% over 10 min, where it was held for another 8 min, and finally decreased linearly to 5% over 10 min, where it was held until the end of a 5-min run. The column temperature was maintained at 25° C., and the injection volume was 10 μL.

### 1.4.8 CMC Determination

A standard pyrene as the fluorescence probe technique was employed to determine the critical micelle concentration (CMC) of QA-M. Hou et al., 2011. Briefly, 1 mL of a 1 mg/mL pyrene solution in acetone was transferred to empty containers and acetone was allowed to subsequently evaporate by gas flow in the dark. A series of QA-M with different polymer concentration were added to each flask to achieve final concentration from 2.5×10⁻⁵ to 3×10⁻² mg/mL. After sonicated for 30 min, the combination of micelles and pyrene were incubated at 60° C. for 1 h. After equilibrated overnight in dark at room temperature, the samples were measured using a Cary Eclipse fluorescence spectrophotometer (Agilent, Calif., USA). The emission wavelength was adjusted to 390 nm, and excitation wavelength 330 nm and 340 nm of pyrene were selected as the detection wavelength. The intensity ratios (I₃₄₄/I₃₃₀) was plotted as a function of logarithm of polymer concentration. The CMC value of QA-M was determined from the intersection of the best-fit lines, which indicated the minimum polymer concentration required for the formation of stable micelles in aqueous medium.

### 1.4.9 Dilution Stability

The dilution stability of the QA-M micelles were investigated by incubating them in PBS (pH=7.4) in 12-60 fold dilutions at 37° C. for 24 hours as previously reported. Valera-Garcia et al., 2018; Zhang et al., 2017. The size distribution and drug entrapment efficiency were determined with methods mentioned above.

### 1.4.10 In Vitro Release

The release behaviors of Q and A from QA-M were investigated by a dialysis method. The QA-M (2 mL) was placed into a preswelled dialysis bag (cutting Mw 8000), which was then immersed into empty lecithin suspension (PC-98T, 100 mg/mL, 100 mL) at 37° C. for 72 hours under stirring at a speed of 100 rpm. The lecithin suspension was formed by film-hydration method followed by sonication and the size of this lecithin suspension was around 100 nm. Sink condition was confirmed by determination of the maximum concentration for free Q and A in the lecithin suspension, which was 0.5 mg/mL and 0.8 mg/mL, respectively. At different time points, 1.0 mL of sample was withdrawn from the release medium, mixed with methanol and measured for the Q and A using the HPLC method mentioned above. The release medium was replenished with an equal volume of fresh medium at 37° C. Sink condition was maintained throughout the experiment. All measurements were performed in triplicate, and the mean values and standard deviations are calculated.

### 1.4.11 Micelles Distribution

To observe the distribution of micelles, DiD-loaded micelles (DiD-M, 150 μg/kg) were prepared as aforementioned and injected into tumor-bearing mice. The mice of DiD-M and PBS-treated groups were sacrificed after 24 h. Major organs and tumors were collected and observed by IVIS imaging. Region-of-interest (ROI) fluorescence intensities of tumors and major organs were detected (n=3).

### 1.4.12 Pharmacokinetics and Tissue Distribution.

Twelve healthy Sprague-Dawley rats (200±20 g) were divided randomly into two groups and fasted overnight before experimentation. Rats in these two groups were injected (i.v.) into the tail vein with a mixed solution of Q and A (QA-F) and QA-M, at 3 mg/kg for Q and 9 mg/kg for A. After injection at designated times (0.0083, 0.0167, 0.033, 0.117, 0.25, 0.5, 1, 2, 4, 8, 12, and 24 h), blood samples (500 μL) were withdrawn from the retro-orbital plexus. The blood samples were centrifuged at 6,000 rpm for 5 min at room temperature, and 200 μL of the separated plasma maintained at −80° C. for analysis.

Fifty μL of the internal standards methanol solution of puerarin (1.0 μg/mL) and 1-naphthyl acetate (0.5 μg/mL), respectively, were added to the 100 μL of serum samples to determine Q and A, respectively. The mixture was vortexed for 5 min. Then 2 mL of ethyl acetate was added to the mixture and vortexed for another 10 min before being centrifuged at 10,000 rpm for 10 min at room temperature to dissolve the drug in the organic solvent. The obtained supernatant was dried under N₂ and resolved in ethanol before it was subjected to ultra-high-performance liquid chromatography (UHPLC)/mass spectrometry (MS) for the detection of Q and A using a TRIPLE QUDA 4500 liquid chromatograph triple quadrupole mass spectrometer equipped with an electrospray ion source in positive mode (AB SCIEX, Framingham, Mass., USA).

Chromatographic separation was determined on a XB-C18 Ultimate UHPLC column (21 mm×50 mm, 1.8 μm, Welch Materials, TX, USA). Gradient elution was done using solvent A (0.1% formic acid solution) and solvent B (acetonitrile). Gradient elution was done at a flow rate of 0.28 mL/min. Initially, 10% organic solvent (acetonitrile containing formic acid solution) was used from 0.01 min to 1 min and increased linearly to 90% in 1 min, where it was held for another 2.7 min, and then decreased to 10% in another 3.3 min, and finally decreased to 10% at 8 min, where it was held until the end of the 8-min run.

The mass spectrometer was operated in positive ion mode within multi-ion reaction monitoring mode. The ion-reaction ratios for quantitative analyses of Q and the internal standard puerarin were m/z 303.1→m/z 229.2 and m/z 416.8→m/z 297.2, respectively. The collision energy of Q and internal standard was 42 V and 43 V, respectively. The ion-reaction ratios for quantitative analyses of A and the internal standard 1-naphthyl acetate were m/z 233.1→m/z 117.1 and m/z 187.2→m/z 145.0, respectively. The collision energy for A and the internal standard was 24 V and 10 V, respectively. Ionization conditions included use of an electrospray ion source with an injection voltage of 5.5 kV, an ion source temperature of 600° C., 50 psi for GS1 and 45 psi for GS2 pressures, and 9 psi for the collision gas pressure.

An orthotopic Ct-26-FL3 colorectal tumor model was established in female BALB/c mice as reported previously. Song et al., 2018. Twenty-four tumor-bearing Balb/c mice were divided randomly into two groups and fasted overnight before experimentation. Mice in these two groups were injected (i.v.) with QA-F and QA-M at 3 mg/kg for Q and 9 mg/kg for A into the tail vein. Another three animals were sacrificed without treatment and their tissues used as blank controls and for preparation of control spiked samples.

Animals were sacrificed in groups of three at 2, 4, 12 and 24 h. Tissue samples were homogenized with saline to 0.2 g/mL. Fifty microliter methanol solution of puerarin 1 μg/mL and 1-naphthyl acetate 500 ng/mL as internals for Q and A, respectively, was added to tissue homogenate. Tissue samples were vortexed for 5 min before the addition of 2 mL ethyl acetate. The mixture was then vortexed for 10 min then centrifuged at 10,000 rpm for 10 min to get supernatant for detection. The obtained supernatant was dried under N₂ and resolved in ethanol before it was subjected to UHPLC/MS mentioned in pharmacokinetic study.

### 1.4.13 Orthotopic Colon Tumor Growth Inhibition Assay

An orthotopic CT26-FL3 colorectal tumor model was established in female Balb/c mice as reported previously. Song et al., 2018. Twenty-eight tumor-bearing mice were divided into seven random groups: PBS, free Q (Q-F), free A (A-F), combination of free Q and A (QA-F), Q-M, A-M and QA-M. Formulations were intravenously injected to mice once every 2 days by total four injections (i.v.) with a Q dose of 3 mg/kg and A dose of 9 mg/kg. Mice in PBS groups were injected with PBS as control. The tumor burden was detected by intraperitoneal (i.p.) injection of 100 μL of D-luciferin (Pierce™, 20 mg/mL) followed by bioluminescent analysis using an IVIS® Kinetics Optical System (Perkin Elmer, Calif.). The tumor growth and body weight of mice were recorded every 2 days. The increment of tumor volume was calculated as luminescence intensities and normalized to the original value on the first day of measurement (Vₜ/V₀). Body weight of mice in each group was documented. Nine days after the last injection, mice were sacrificed and the tumor, heart, liver, spleen, lung and kidney tissues were removed and used for the present study. Blood samples were collected from the orbital plexus into heparinized tubes and then centrifuged at 5,000 rpm for five minutes to separate the plasma. One portion of tumor, spleen and lymph nodes were collected for flow cytometric analysis. One portion of the tumor was fixed in 4% formalin, paraffin-embedded and sectioned for the terminal deoxynucleotidyl transferase-mediated nick end labeling (TUNEL) assay, immunofluorescence staining and hematoxylin and eosin (H&E) staining. One portion of tumor was stored at −80° C. for western blotting and RT-PCR assay.

### 1.4.14 H&E Staining

The tumors were fixed in 4% formalin, paraffin-embedded and sectioned for hematoxylin and eosin (H&E) staining. Apoptosis, metastasis and toxicity were determined by H&E staining and photographed by optical microscopy.

### 1.4.15 TUNEL Assay

TUNEL assays were performed as recommended by the manufacturer (Promega, Madison, Wis., USA). Cell nuclei were staining by DAPI mounting medium. The samples were analyzed by Olympus IX81 inverted microscope and quantified by Image J software.

### 1.4.16 Flow Cytometric Analysis

Single-cell suspensions of tumor and spleen were processed and collected as previously described. Song et al., 2018. Splenocytes, lymphocytes and tumor-infiltrating leukocytes (TILs) were analyzed by flow cytometry after immunofluorescence staining. Cells were stained with antibodies conjugated with fluorophores (Table 2). All antibodies were purchased from Biolegend (San Diego, Calif.) or Abcam (Cambridge, Mass.). All samples were analyzed by using an 18-color flow cytometer (LSR II, BD Biosciences, Calif.) and data were analyzed with FlowJo 8.6 software (TreeStar).

TABLE 2
Antibodies list

| Antibodies | Company | Application |
|---|---|---|
| Alexa Fluor ® 647 Anti-CD3 antibody | BioLegend | IF |
| APC/Alexa Fluor ® 594 anti-mouse CD4 | BioLegend | Flow |
| antibody | | |
| eFluro 450 anti-mouse CD8a antibody | BioLegend | Flow |
| PE/Cy7/Alexa Fluor ® 594 anti-mouse | BioLegend | Flow |
| CD11c antibody | | |
| APC anti-mouse CD62L antibody | BioLegend | Flow |
| Alexa Fluor ® 488 anti-mouse CD44 | BioLegend | Flow |
| antibody | | |
| PE anti-mouse TLR4 antibody | BioLegend | Flow |
| APC/Cy7 anti-mouse/human CD11b | BioLegend | Flow |
| antibody | | |
| Brilliant Violet 510 ™ anti-mouse/human | BioLegend | Flow |
| CD11b antibody | | |
| Alexa Fluor ® 594 anti-Gr1 antibody | BioLegend | Flow |
| FITC anti-mouse NK-1.1 antibody | BioLegend | Flow |
| APC anti-mouse IL-12 | BioLegend | Flow |
| PE-Cy7 anti-mouse IFN-γ antibody | BioLegend | Flow |
| APC/Cy7 anti-mouse CD86 | BioLegend | Flow |
| Brilliant Violet 605 ™ anti-mouse CD274 | BioLegend | Flow |
| (B7-H1, PD-L1) Antibody | | |
| Alexa Fluor 488 Anti-Foxp3 | BioLegend | Flow |
| Brilliant Violet 421 ™ anti-mouse I-A/I-E | BioLegend | Flow |
| PE mouse anti-mouse I-A [b] | BD | Flow |
| FITC Anti-CD45 | BioLegend | Flow |
| Anti-CRT antibody | Abcam | Flow/WB/IF |
| Anti-HMGB1 antibody | Abcam | IF |
| Alexa Fluor ® 488 Goat Anti-Rabbit (IgG) | Abcam | IF |
| Bcl-2 (50E3) Rabbit mAb | CST | WB |
| Bcl-xL (54H6) Rabbit mAb | CST | WB |
| Phospho-AMPKα (Thr172) Antibody | CST | WB |
| mTOR (7C10) Rabbit mAb | CST | WB |
| Phospho-mTOR (Ser2448) (D9C2) XP ® | CST | WB |
| Rabbit mAb | | |
| GAPDH (D16H11) XP ® Rabbit mAb | CST | WB |

IF: immunofluorescence.
Flow: flow cytometry.
WB: western blot.

### 1.4.17 Quantitative Real-Time Polymerase Chain Reaction (RT-PCR)

Total RNA from the tumor tissues was extracted using an RNeasy Microarray Tissue Mini Kit (Qiagen). cDNA was reverse-transcribed using the iScript™ cDNA Synthesis Kit (BIO-RAD). cDNA (150 ng) was amplified by using the TaqMan™ Gene Expression Master Mix for RT-qPCR (ThermoFisher). GAPDH was used as the endogenous control. RT-PCR primers are listed in Table 3 with specific catalog numbers. A 7500 Real-Time PCR System was used to conduct the reactions and the data were analyzed by 7500 software.

TABLE 3
Primer list for real-time PCR

| Primers | Applied Biosystems |
|---|---|
| Mouse IFN-γ | Mm01168134_m1 |
| Mouse TNF-α | Mm00443260_g1 |
| Mouse TGF-β | Mm01178820_m1 |
| Mouse IL10 | Mm01288386_m1 |
| Mouse CXCL9 | Mm00434946_m1 |
| Mouse CCL2 | Mm00441242_m1 |
| Mouse IL-1β | Mm00434228_m1 |
| Mouse GAPDH | Mm99999915_g1 |

### 1.4.18 Immunofluorescence Staining

After deparaffinization, antigen retrieval and permeabilization, samples were blocked in 5% BSA at room temperature for 1 h. Anti-CD3 conjugated with Alexa Fluor 647 (Biolegend, San Diego, US) was added to the slides at 4° C. overnight. Then the nuclei were counterstained by Antifade Mounting Medium with DAPI. Samples were observed under Olympus IX81 inverted microscope and quantified by Image J software.

### 1.4.19 Western Blot Analysis

Proteins were extracted and quantified by the Pierce BCA Protein Assay Kit (Thermo Scientific, USA). Forty micrograms of proteins were electrophoretically separated using NuPAGE 4-12% Bis-Tris SDS-PAGE gel and transferred to polvinylidene fluoride membrane (PVDF; Thermo Scientific). PVDF membranes with proteins were blocked by 5% BSA in PBS Tween 20 solution (Fisher Scientific, Faith Lawn, N.J., USA) for 1 h. Membranes were then incubated at 4° C. for overnight by primary antibodies (1:1000 dilution, Bcl-2, Bcl-xL, p-AMPKα, mTOR, p-mTOR, CRT and GAPDH) followed by incubation with the horseradish peroxidase (HRP)-conjugated secondary antibody anti-rabbit IgG (Cell Signal Technology, Danvers, Mass., USA) for 1 h at room temperature. The membranes were washed and captured with ChemiDoc XRS+ imaging system (Bio-Rad, CA, USA). GAPDH was used as a loading control. Image J software (National Institutes of Health) was used to semi-quantify the mean grey value and normalized to that of GAPDH.

### 1.4.20 Long-Term Anti-Tumor Immune-Memory Effects

A total of 1×10⁶ CT26-FL3 cells were inoculated orthotopically into twenty five 6-week-old female Balb/C mice (Janvier, Charles River) and i.v. injected with PBS and QA-M (3 mg/kg for Q, 9 mg/kg for A) as aforementioned. Anti-mouse CD8α, anti-mouse CD4 and anti-rat IgG (200 μg per mice, i.p.) were given one day before the QA-M injection for 3 injection in total at every three day. Song et al., 2018. The tumor volumes were monitored and recorded using IVIS system every other day.

Ten mice were inoculated with 1×10⁶ CT26-FL3 cells to establish orthotopic colorectal murine model. Four days after the last injection, 1×10⁶ 4 T1 cells were inoculated into the lower right flank, whereas 1×10⁶CT26-FL3 cells were inoculated into the contralateral flank on the same day. The tumor volume from both side of mice were recorded.

### 1.4.20 Therapeutic Efficacy in Orthotopic Breast Cancer Model

Twelve Balb/c female mice were inoculated with 4T1 cells (1×10⁶ per mouse) at mammary gland to create orthotopic breast-tumor model. When the tumor volume reached 100 mm³, the mice were divided into three groups: PBS, QA-F and QA-M. Formulations were administered to the mice once every other days by four injections (i.v.) with a Q dose of 3 mg/kg and A dose of 9 mg/kg. Mice in the control group were administered PBS only. The tumor volume of the mice was measured every other days and calculated using Formula (2):

V=(W²×L)/2 (2)

where V is the tumor volume, W is the smaller perpendicular diameter, and L is the larger perpendicular diameter. Tumor weight were measured at the end of experiment and imaged.

### 1.4.20 Statistics Assay

All the results are presented as the mean±standard deviation (SD). The Student's t-test and one-way analysis of variance were used to evaluate significance, and p<0.05 was considered significant.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
American Cancer Society. Colorectal Cancer Facts & FIGS. 2017-2019. Atlanta: American Cancer Society. 2017.
Weitz, J.; Koch, M.; Debus, J.; Höhler, T.; Galle, R. R.; Büchler, M. W. Colorectal Cancer. The Lancet. 2005, 365, 8-14.
McKeown, E.; Nelson, D. W.; Johnson, E. K.; Maykel, J. A.; Stojadinovic, A.; Nissan, A.; Avital, I.; Brücher, B. L.; Steele, S. R. Current Approaches and Challenges for Monitoring Treatment Response in Colon and Rectal cancer. J Cancer. 2014, 5, 31-43.
Birendra, K. C.; Hwang, J. J.; Farhangfar, C. J.; Chai, S. J. Advances in Immunotherapy in the Treatment of Colorectal Cancer. AJHO. 2017, 13, 4-8.
Goodwin, T. J.; Huang, L. Investigation of Phosphorylated Adjuvants Co-encapsulated With A Model Cancer Peptide Antigen for the Treatment of Colorectal Cancer and Liver Metastasis. Vaccine. 2017, 35, 2550-2557.
Alev, M.; Egenberger, L.; Mühleisen, L.; Weigel, B.; Frey, B.; Friedrich, R. P.; Pöttier, M.; Alexiou, C.; Janko, C. Targeting of Drug-loaded Nanoparticles to Tumor Sites Increases Cell Death and Release of Danger Signals. J Control Release. 2018, 285, 67-80.
Gajewski, T. F.; Schreiber, H.; Fu, Y. X. Innate and Adaptive Immune Cells in the Tumor Microenvironment. Nat Immunol. 2013, 14, 1014-1022.
Wellenstein, M. D.; de Visser, K. E. Cancer-Cell-Intrinsic Mechanisms Shaping the Tumor Immune Landscape. Immunity. 2018, 48, 399-416.
Duan, H. F. Novel Therapeutic Strategies for Solid Tumor Based On Body's Intrinsic Antitumor Immune System. Cell Physiol Biochem. 2018, 47, 441-457.
Marin-Acevedo, J. A.; Soyano, A. E.; Dholaria, B.; Knutson, K. L.; Lou, Y. Cancer Immunotherapy Beyond Immune Checkpoint Inhibitors. J Hematol Oncol. 2018, 11, 8.
Showalter, A.; Limaye, A.; Oyer, J. L.; Igayashi, R.; Kittipatarin, C.; Copik, A. J.; Khaled, A. R. Cytokines in Immunogenic Cell Death: Applications for Cancer Immunotherapy. Cytokine. 2017, 97, 123-132.
Pfirschke, C.; Engblom, C.; Rickelt, S.; Cortez-Retamozo, V.; Garris, C.; Pucci, F.; Yamazaki, T.; Poirier-Colame, V.; Newton, A.; Redouane, Y.; et al. Immunogenic Chemotherapy Sensitizes Tumors to Checkpoint Blockade Therapy. Immunity. 2016, 44, 343-354.
Song, W. T.; Shen, L. M.; Wang, Y.; Liu, Q.; Goodwin, T. J.; Li, J.; Dorosheva, O.; Liu, T.; Liu, R. H.; Huang, L. Synergistic and Low Adverse Effect Cancer Immunotherapy by Immunogenic Chemotherapy and Locally Expressed PD-L1 Trap. Nat Commun. 2018, 9, 2237.
Gilabert-Oriol, R.; Ryan, G. M.; Leung, A. W. Y.; Firmino, N. S.; Bennewith, K. L.; Bally, M. B. Liposomal Formulations to Modulate the Tumour Microenvironment and Antitumour Immune Response. Int J Mol Sci. 2018, 19, 2922.
Hodge, J. W.; Garnett, C. T.; Farsaci, B.; Palena, C.; Tsang, K. Y.; Ferrone, S.; Gameiro, S. R. Chemotherapy-induced Immunogenic Modulation of Tumor Cells Enhances Killing by Cytotoxic T Lymphocytes and Is Distinct from Immunogenic Cell Death. Int J Cancer. 2013, 133, 624-636.
Suryadevara, C. M.; Riccione, K.; Sampson, J. H. Immunotherapy Gone Viral: Bortezomib and oHSV Enhance Antitumor NK cell Activity. Clin Cancer Res. 2017, 22, 5164-5166.
Kono, K.; Mimura, K.; Kiessling, R Immunogenic Tumor Cell Death Induced by Chemoradiotherapy: Molecular Mechanisms and A Clinical Translation. Cell Death Dis. 2013, 4, 688.
Zhang, J.; Zhang, P.; Zou, Q.; Li, X.; Fu, J. J.; Luo, Y.; Liang, X. L.; Jin, Y. Co-Delivery of Gemcitabine and Paclitaxel in CRGD-modified Long Circulating Nanoparticles with Asymmetric Lipid Layers for Breast Cancer Treatment. Molecules. 2018, 23, 2906.
Gubin, M. M.; Schreiber, R. D. The Odds of Immunotherapy Success. Science. 2015, 350, 158-159.
Tesniere, A.; Schlemmer, F.; Boige, V.; Kepp, O.; Martins, I.; Ghiringhelli, F.; Aymeric, L.; Michaud, M.; Apetoh, L.; Barault, L.; et al Immunogenic Death of Colon Cancer Cells Treated With Oxaliplatin. Oncogene. 2016, 29, 482-491.
Obeid, M.; Tesniere, A.; Ghiringhelli, F.; Fimia, G. M.; Apetoh, L.; Perfettini, J. L.; Castedo, M.; Mignot, G.; Panaretakis, T.; Casares, N.; et al. Calreticulin Exposure Dictates the Immunogenicity of Cancer Cell Death. Nat Med. 2007, 13, 54-61.
Martins, I.; Wang, Y.; Michaud, M.; Ma, Y.; Sukkurwala, A. Q.; Shen, S.; Kepp, O.; Métivier, D.; Galluzzi, L. Perfettini, J. L.; et al. Molecular Mechanisms of ATP Secretion During Immunogenic Cell Death. Cell Death Differ. 2014, 21, 79-91.

Kroemer, G.; Galluzzi, L.; Kepp, O.; Zitvogel, L Immunogenic Cell Death in Cancer Therapy. Ann Rev Immunol. 2013, 31, 51-72.
Lu, J.; Liu, X.; Liao, Y. P.; Salazar, F.; Sun, B.; Jiang, W.; Chang, C. H.; Jiang, J.; Wang, X.; Wu, A. M.; et al. Nano-enabled Pancreas Cancer Immunotherapy Using Immunogenic Cell Death and Reversing Immunosuppression. Nat Commun. 2017, 8, 1811.
Crawford, J.; Dale, D. C.; Lyman, G. H. Chemotherapy-induced Neutropenia: Risks, Consequences, and New Directions for Its Management. Cancer. 2004, 100, 228-237.
Liu, Q.; Chen, F. Q.; Hou, L.; Shen, L. M.; Zhang, X. Q.; Wang, D. G.; Huang, L. Nanocarrier-mediated Chemo-Immunotherapy Arrested Cancer Progression and Induced Tumor Dormancy in Desmoplastic Melanoma. ACS Nano. 2018, 12, 7812-7825.
Wang, S. F.; Liu, K. C.; Wang, X. M.; He, Q. X.; Chen, X. Q. Toxic Effects of Celastrol on Embryonic Development of Zebrafish (Danio rerio). Drug Chem Toxicol. 2011, 34, 61-65.
Cascão, R.; Eonseca, J. E.; Moita, L. F. Celastrol: A Spectrum of Treatment Opportunities in Chronic Diseases. Front Med (Lausanne). 2017, 4, 69.
Ward, A. B.; Mir, H.; Kapur, N.; Gales, D. N.; Carriere, P. P.; Singh, S. Quercetin Inhibits Prostate Cancer by Attenuating Cell Survival and Inhibiting Anti-Apoptotic Pathways. World J Surg Oncol. 2018a, 16, 108.
Feng, J.; Song, D.; Jiang, S.; Yang, X.; Ding, T.; Zhang, H.; Luo, J.; Liao, J. Y in, Q. Quercetin Restrains TGF-β1-induced Epithelial-Mesenchymal Transition by Inhibiting Twist1 and Regulating E-Cadherin Expression. Biochem Biophys Res Commun. 2018, 498, 132-138.
Rockenbach, L.; Bavaresco, L.; Fernandes F. P.; Cappellari, A. R.; Barrios, C. H.; Bueno Morrone, F.; Oliveira Battastini, A. M. Alterations in the Extracellular Catabolism of Nucleotides Are Involved in the Antiproliferative Effect of Quercetin in Human Bladder Cancer T24 cells. Urol Oncol. 2013, 31, 1204-1211.
Egert, S.; Wolffram, S.; Bosy-Westphal, A.; Boesch-Saadatmandi, C.; Wagner, A. E.; Frank, J.; Rimbach, G.; Mueller, M. J. Daily Quercetin Supplementation Dose-Dependently Increases Plasma Quercetin Concentrations in Healthy Humans. J Nutr. 2008, 138, 1615-1621.
Ward, A. B.; Mir, H.; Kapur, N.; Gales, D. N.; Carriere, P. P.; Singh, S. Quercetin Inhibits Prostate Cancer by Attenuating Cell Survival and Inhibiting Anti-Apoptotic Pathways. World J Surg Oncol. 2018b, 16, 108.
Chun, J.; Choi, R. J.; Khan, S.; Lee, D. S.; Kim, Y. C.; Nam, Y. J.; Lee, D. U.; Kim, Y. S. Alantolactone Suppresses Inducible Nitric Oxide Synthase and Cyclooxygenase-2 Expression by Down-regulating NF-KB, MAPK and AP-1 via the MyD88 Signaling Pathway in LPS-activated RAW 264.7 Cells. Int Immunopharmacol. 2012, 14, 375-383.
Rasul, A.; Khan, M.; Ali, M.; Li, J.; Li, X. Targeting Apoptosis Pathways in Cancer with Alantolactone and Isoalantolactone. Scientific World Journal, 2013, 248532.
Chun, J.; Li, R. J.; Cheng, M. S.; Kim, Y. S. Alantolactone Selectively Suppresses STAT3 Activation and Exhibits Potent Anticancer Activity in MDA-MB-231 Cells. Cancer Lett. 2015, 357, 393-403.
Khan, M.; Yi, F.; Rasul, A.; Li, T.; Wang, N.; Gao, H.; Gao, R.; Ma, T. Alantolactone Induces Apoptosis in Glioblastoma Cells via GSH Depletion, ROS Generation, and Mitochondrial Dysfunction. IUBMB Life. 2012, 64, 783-794.
Kawano, M.; Tanaka, K.;
Itonaga, I.; Iwasaki, T.; Miyazaki, M.; Ikeda, S.; Tsumura, H. Dendritic Cells Combined With Doxorubicin Induces Immunogenic Cell Death and Exhibits Antitumor Effects for Osteosarcoma. Oncol Lett. 2016, 11, 2169-2175.
Liu, Y.; Gong, W.; Yang, Z. Y.; Zhou, X. S.; Gong, C.; Zhang, T. R.; Wei, X.; Ma, D.; Ye, F.; Gao, Q. L. Quercetin Induces Protective Autophagy and Apoptosis Through ER Stress via the P-STAT3/Bcl-2 Axis in Ovarian Cancer. Apoptosis. 2017, 22, 544-557.
Miao, L.; Guo, S. T.; Zhang, J.; Kim, W. Y.; Huang, L. Nanoparticles with Precise Ratiometric Co-Loading and Co-Delivery of Gemcitabine Monophosphate and Cisplatin for Treatment of Bladder Cancer. Adv Funct Mater. 2014, 24, 6601-6611.
Jin, Y.; Wu, Z.; Li, C.; Zhou, W.; Shaw, J. P.; Baguley, B. C.; Liu, J.; Zhang, W. Optimization of Weight Ratio for DSPE-PEG/TPGS Hybrid Micelles to Improve Drug Retention and Tumor Penetration. Pharm Res. 2018, 35, 13.
Sezgin, Z.; Yüksel, N.; Baykara, T. Preparation and Characterization of Polymeric Micelles for Solubilization of Poorly Soluble Anticancer Drugs. Eur J Pharm Biopharm. 2006, 64, 261-268.
Mi, Y.; Liu, Y.; Feng, S. S. Formulation of Docetaxel by Folic Acid-conjugated-α-Tocopheryl Polyethylene Glycol Succinate 2000 (Vitamin E TPGS) Micelles for Targeted and Synergistic Chemotherapy. Biomaterials, 2011, 32, 4058-4066.
Parveen, S.; Sahoo, S. K. Long Circulating Chitosan/PEG Blended PLGA Nanoparticle for Tumor Drug Delivery. Eur J Pharmacol. 2011, 670, 372-383.
Zhang, Y.; Schwerbrock, N. M.; Rogers, A. B.; Kim, W. Y.; Huang, L. Codelivery of VEGF siRNA and Gemcitabine Monophosphate in A Single Nanoparticle Formulation for Effective Treatment of NSCLC. Mol Ther. 2013, 21, 1559-1569.
Lee, T. J.; Kim, O. H.; Kim, Y. H.; Lim, J. H.; Kim, S; Park, J. W.; Kwon, T. K. Quercetin Arrests G2/M Phase and Induces Caspase-Dependent Cell Death in U937 Cells. Cancer Lett. 2006, 240, 234-42.
Zhao, P.; Pan, Z.; Luo, Y.; Zhang, L.; Li, X.; Zhang, G.; Zhang, Y.; Cui, R.; S un, M.; Zhang, X. Alantolactone Induces Apoptosis and Cell Cycle Arrest on Lung Squamous Cancer SK-MES-1 Cells. J Biochem Mol Toxicol. 2015, 29, 199-206.
Wu, C. J.; Tsai, Y. T.; Lee, I. J.; Wu, P. Y.; Lu, L. S.; Tsao, W. S.; Huang, Y. J.; Chang, C. C.; Ka, S. M.; Tao, M. H. Combination of Radiation and Interleukin 12 Eradicates Large Orthotopic Hepatocellular Carcinoma Through Immunomodulation of Tumor Microenvironment. Oncoimmunology. 2018, 7, 1477459.
Fridman, W. H.; Pagés, F.; Sautës-Fridman, C.; Galon, J. The Immune Contexture in Human Tumours: Impact on Clinical Outcome. Nat Rev Cancer. 2012, 12, 298-306.
Sakaguchi, S.; Miyara, M.; Costantino, C. M.; Hafler, D. A. FOXP3+ Regulatory T Cells in the Human Immune System. Nat Rev Immunol. 2010, 10, 490-500.
Xu, J.; Escamilla, J.; Mok, S.; David, J.; Priceman, S.; West, B.; Bollag, G.; M cBride, W.; Wu, L. CSF1R Signaling Blockade Stanches Tumor-infiltrating Myeloid Cells and Improves the Efficacy of Radiotherapy in Prostate Cancer. Cancer Res. 2013, 73, 2782-2794.
Li, T. T.; Ogino, S.; Qian, Z. R. Toll-Like Receptor Signaling in Colorectal Cancer: Carcinogenesis to Cancer Therapy. World J Gastroenterol. 2014, 20, 17699-17708.
Yesudhas, D.; Gosu, V.; Anwar, M. A.; Choi, S. Multiple Roles of Toll-Like Receptor 4 in Colorectal Cancer. Front Immunol. 2014, 5, 334.
Wang, D.; DuBois, R. N. The Role of Anti-Inflammatory Drugs in Colorectal Cancer. Ann Rev Med. 2013, 64, 131-144.
Li, Y.; Yao, J. Y.; Han, C. Y.; Yang, J. X.; Chaudhry, M. T.; Wang, S. N.; Liu, H. N.; Yin, Y. L. Quercetin, Inflammation and Immunity. Nutrients, 2016, 8, 167.
Wang, X.; Yu, Z. L.; Wang, C.; Cheng, W.; Tian, X. G.; Huo, X. K.; Wang, Y.; Sun, C. P.; Fen, L.; Xing, J. S.; et al. Alantolactone, A Natural Sesquiterpene Lactone, Has Potent Antitumor Activity Against Glioblastoma by Targeting IKKβ Kinase Activity and Interrupting NF-κB/COX-2-mediated Signaling Cascades. J Exp Clin Cancer Res. 2017, 36, 93.
Sage, P. T.; Schildberg, F. A.; Sobel, R. A.; Kuchroo, V. K.; Freeman, G. J.; Sharpe, A. H.; Dendritic Cell PD-L1 Limits Autoimmunity and Follicular T Cell Differentiation and Function. J Immunol. 2018, 200, 2592-2602.
Palucka, K.; Banchereau, J. Cancer Immunotherapy via Dendritic Cells. Nat Rev Cancer. 2012, 12, 265-277.
Han, J. H.; Suh, C. H.; Jung, J. Y.; Ahn, M. H.; Han, M. H.; Kwon, J. E.; Yim, H.; Kim, H. A. Elevated Circulating Levels of the Interferon-γ-induced Chemokines Are Associated With Disease Activity and Cutaneous Manifestations in Adult-Onset Still's Disease. Sci Rep. 2017, 7, 46652.
Le, P. C.; Génin, P.; Baines, M. G.; Hiscott, J. Interferon Activation and Innate Immunity. Rev Immunogenet. 2000, 2, 374-386.
Wang, N.; Zhang, Q.; Luo, L.; Ning, B.; Fang, Y. β-Asarone Inhibited Cell Growth and Promoted Autophagy via P53/Bcl-2/Bclin-1 and P53/AMPK/mTOR Pathways in Human Glioma U251 cells. J Cell Physiol. 2018, 233, 2434-2443.
Kim, J; Kundu, M.; Viollet, B.; Guan, K. L. AMPK and mTOR Regulate Autophagy Through Direct Phosphorylation of Ulk1. Nat Cell Biol. 2011, 13, 132-141.
Kim, J; Yang, G.; Kim, Y.; Kim, J.; Ha, J. AMPK Activators: Mechanisms of Action and Physiological Activities. Exp Mol Med. 2016, 48, 224.
Rivera Rivera, A.; Castillo-Pichardo, L.; Gerena, Y.; Dharmawardhane, S. Anti-Breast Cancer Potential of Quercetin via the Akt/AMPK/Mammalian Target of Rapamycin (mTOR) Signaling Cascade. PLoS One, 2016, 11, 157251.
Ahn, J.; Lee, H.; Kim, S.; Park, J.; Ha, T. The Anti-Obesity Effect of Quercetin is Mediated by the AMPK and MAPK Signaling Pathways. Biochem Biophys Res Commun. 2008, 373, 545-549.
Zheng, J.; Ramirez, V. D. Inhibition of Mitochondrial Proton F0F1-ATPase/ATP Synthase by Polyphenolic Phytochemicals. Br J Pharmacol. 2000, 130, 1115-1123.
Zhang, J.; Miao, L.; Guo, S. T.; Zhang, Y.; Zhang, L.; Satterlee, A.; William, K.; Huang, L. Synergistic Anti-tumor Effects of Combined Gemcitabine and Cisplatin Nanoparticles in A Stroma-Rich Bladder Carcinoma Model. J Control Release. 2014, 182, 90-96.
Chou, T. C.; Talalay, P. Quantitative Analysis of Dose-Effect Relationships: the Combined Effects of Multiple Drugs or Enzyme Inhibitors. Adv Enzyme Regul. 1984, 22, 27-55.
Hou, L.; Fan, Y.; Yao, J.; Zhou, J. P.; Li, C. C.; Fang, Z. J.; Zhang, Q. Low Molecular Weight Heparin-All-Trans-Retinoid Acid Conjugate as A Drug Carrier for Combination Cancer Chemotherapy of Paclitaxel and All-Trans-Retinoid Acid. Carbohydr Polyme. 2011, 86, 1157-1166.
Varela-Garcia, A.; Concheiro, A.; Alvarez-Lorenzo, C. Soluplus Micelles for Acyclovir Ocular Delivery: Formulation and Cornea and Sclera Permeability. Int J Pharm. 2018, 552, 39-47.
Zhang, T.; Zhou, S. L.; Liu, Y.; Luo, X.; Di, D. H.; Song, Y. Z.; Liu, X. R.; Deng, X. H. Polysialic Acid and Pluronic F127 Mixed Polymeric Micelles of Docetaxel As New Approach for Enhanced Antitumor Efficacy. Drug Dev Ind Pharm. 2017, 43, 1827-1835.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A micellar formulation comprising a synergistically effective amount of quercetin and alantolactone, or derivatives thereof, for treating a cancer.

2. The micellar formation of claim 1, wherein the quercetin and alantolactone are present in the micellar formulation in a molar ratio selected from the group consisting of about 1:13 quercetin:alantolactone (mol/mol), about 1:7 quercetin:alantolactone (mol/mol), and about 1:4 quercetin:alantolactone (mol/mol).

3. The micellar formulation of claim 1, wherein the quercetin and alantolactone are present in the micellar formulation in a molar ratio of about 1:4 quercetin:alantolactone (mol/mol).

4. The micellar formulation of claim 1, wherein the micellar formulation comprises a combination of 1,2-distearoyl-sn-glycero-3-phosphoethanolamine-N-methoxy-poly(ethylene glycol 2000) (DSPE-PEG2000) and D-α-Tocopherol polyethylene glycol succinate (TPGS).

5. The micellar formulation of claim 1, wherein the micellar formulation comprises spherical particles.

6. The micellar formulation of claim 5, wherein the spherical particles have a diameter of about 20 nm.

7. The micellar formulation of claim 5, wherein the micellar formulation has a zeta potential of about −0.3±0.1 mV.

8. The micellar formulation of claim 1, wherein the micellar formulation has an encapsulation efficiency of greater than about 90% for quercetin and alantolactone.

9. The micellar formulation of claim 1, wherein the micellar formulation has a critical micelle concentration (CMC) of about 0.003 mg/mL.

10. A method for treating a cancer in a subject in need of treatment thereof, the method comprising administering to the subject a therapeutically effective amount of a micellar formulation of any of claims 1-9 to treat the cancer.

11. The method of claim 10, wherein the cancer is selected from the group consisting of colorectal cancer, breast cancer, pancreatic cancer, cervical cancer, prostate cancer, and lymphoma.

12. The method of claim 11, wherein the colorectal cancer is microsatellite-stable colorectal cancer.

13. The method of claim 10, wherein administration of a synergistically effective amount of quercetin and alantolactone induces immunogenic cell death (ICD) and/or induces cancer cell apoptosis.

14. The method of claim 10, wherein administration of a synergistically effective amount of quercetin and alantolactone inhibits tumor growth and/or progression.

15. The method of claim 10, wherein the administration of a synergistically effective amount of quercetin and alantolactone reduces a percentage of immune cells in a tumor microenvironment of the cancer.

16. The method of claim 15, wherein the immune cells in the tumor microenvironment of the cancer are selected from the group consisting of myeloid-derived suppressor cells (MDSCs) and T regulatory cells (Tregs).

17. The method of claim 10, wherein the administration of a synergistically effective amount of quercetin and alantolactone inhibits tumor-promoting inflammation in one or more cells.

18. The method of claim 10, wherein the administration of a synergistically effective amount of quercetin and alantolactone reduces Toll-like receptor 4 positive (TLR4⁺) expression in one or more cancer cells.

19. The method of claim 10, wherein the administration of a synergistically effective amount of quercetin and alantolactone reduces PD-L1 expression on one or more cancer cells.

20. The method of claim 10, wherein the administration of a synergistically effective amount of quercetin and alantolactone reduces secretion of immune-suppressive cytokines in one or more cancer cells.

21. The method of claim 20, wherein the immune-suppressive cytokines are selected from the group consisting of IL-10, TGF-β, IL-1β, and CCL2.

22. The method of claim 10, wherein the administration of a synergistically effective amount of quercetin and alantolactone activates one or more tumor-infiltrating immune cells in a cancer tumor.

23. The method of claim 22, wherein the one or more tumor-infiltrating immune cells comprises one or more CRT⁺ cells.

24. The method of claim 23, wherein the one or more CRT⁺ cells are selected from the group consisting of a CD3⁺ T cell, a CD8⁺ T cell, and a CD4⁺ T cell.

25. The method of claim 10, wherein the administration of a synergistically effective amount of quercetin and alantolactone increases expression of a level of costimulatory signal (MHC class II and CD86) on one or more dendritic cells.

26. The method of claim 10, wherein the administration of a synergistically effective amount of quercetin and alantolactone increases a presence of natural killer (NK) cells.

27. The method of claim 10, wherein the administration of a synergistically effective amount of quercetin and alantolactone increases IFN-γ production from CD4⁺ and CD8⁺ T cells in a tumor comprising the cancer.

28. The method of claim 10, wherein the administration of a synergistically effective amount of quercetin and alantolactone activates T cells.

29. The method of claim 10, wherein the administration of a synergistically effective amount of quercetin and alantolactone induces higher levels of IL-12 and IFN-γ in a tumor comprising the cancer.

30. The method of claim 10, wherein the administration of a synergistically effective amount of quercetin and alantolactone increases the expression of CXCL9 in one or more cancer cells.

31. The method of claim 10, wherein the administration of a synergistically effective amount of quercetin and alantolactone increases the secretion of tumor necrosis factor alpha (TFN-α) in one or more cancer cells.

32. The method of claim 10, wherein the administration of a synergistically effective amount of quercetin and alantolactone down-regulates suppressive immune cells and cytokines.

33. The method of claim 10, wherein the administration of a synergistically effective amount of quercetin and alantolactone up-regulates immuno-active cells and cytokines.

34. The method of claim 10, wherein the administration of a synergistically effective amount of quercetin and alantolactone increases the expression of phosphor-AMP-activated protein kinase α (p-AMPKα) protein in one or more cancer cells.

35. The method of claim 10, wherein the administration of a synergistically effective amount of quercetin and alantolactone decreases the expression of mammalian target of rapamycin (mTOR) and phospho-mTOR (p-mTOR) in one or more cancer cells.

36. The method of claim 10, wherein the administration of a synergistically effective amount of quercetin and alantolactone inhibits Bcl-2 to induce cell apoptosis, thereby promoting autophagy.

37. The method of claim 10, wherein the administration of a synergistically effective amount of quercetin and alantolactone produces p-AMPK and suppresses mTOR and p-mTOR, thereby promoting autophagy.

38. The method of claim 10, wherein the administration of a synergistically effective amount of quercetin and alantolactone activating innate immune response in tumors, thereby inducing the activation of an adaptive immune response and inhibiting tumor growth.

39. The method of claim 10, wherein the administration of a synergistically effective amount of quercetin and alantolactone recruiting tumor-specific memory T cells.

40. The method of claim 39, wherein the memory T cells include CD8⁺ and CD4⁺.
